(19) United States
(12) Patent Application Publication
(10) Pub. No.: US 2023/0190614 A1
(43) Pub. Date: Jun. 22, 2023

(54) SELF-TANNING COMPOSITIONS CONTAINING AN ALKYL SULFONATE AND METHODS THEREOF

(71) Applicant: Kao Corporation, Tokyo (JP)

(72) Inventors: Jordan CORDEIRO, Erlanger, KY (US); Kotatsu BITO, Cincinnati, OH (US)

(73) Assignee: Kao Corporation, Tokyo (JP)

(21) Appl. No.: 17/996,391

(22) PCT Filed: Apr. 28, 2021
(86) PCT No.: PCT/US2021/029677

§ 371 Date: Oct. 17, 2022

Related U.S. Application Data
(60) Provisional application No. 63/017,034, filed on Apr. 29, 2020.

(51) Int. Cl. A61K 8/60; A61K 8/46; A61K 8/34; A61Q 19/04

(52) U.S. Cl. CPC A61K 8/60; A61K 8/466; A61K 8/34; A61Q 19/04; A61K 2800/57

(57) ABSTRACT

A topical skincare composition that includes (A) a reducing sugar, (B) an optionally substituted C₈₋₁₂ alkyl sulfonate, and (C) a carrier. The composition may further include an aromatic sulfonate. A method for enhancing color appearance (e.g., hue angle, lightness, saturation) of skin, which involves applying the topical skincare composition onto the skin is also specified. The topical skincare composition can provide a deep, rich, long lasting artificial tan with customizable hue preservation or adjustment when applied onto the skin.

### BACKGROUND OF THE INVENTION

### Field of the Invention

The present invention relates to topical skincare compositions, specifically topical skincare compositions that include (A) a reducing sugar, (B) an alkyl sulfonate, and (C) a carrier, as well as methods of self-tanning using the topical skincare compositions.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Consumers around the world enjoy spending time in the sun for a variety of reasons including outdoor recreation, sports, and sun tanning skin darkening). Unfortunately, exposure to UV radiation has known potential to promote skin cancer. Furthermore, partial exposure to UV radiation can lead to uneven skin tone across the body. Health and aesthetic incentives therefore exist for alternative means to achieve natural-looking, even skin tone while avoiding the dangers of sun exposure.

Despite of the known risks, sunbathing remains the most common method to darken skin in the US and the EU. Sunless tanners and glow moisturizers are also common methods, however many consumers are not satisfied with these products, as the colors are often perceived as unnatural, insufficiently dark, or rapidly fading. For example, many conventional self-tanning products containing dihydroxyacetone (PHA) tanning agent produce an undesirable orange hue. In addition to skin darkening, some customers prefer to preserve their skin's original hue, while others desire a hue shift to more aesthetically pleasing bronze/red tone.

Several skin tanning formulations have been reported that utilize azole compounds, pigments (e.g., carmine), crosslinked cationic copolymers, and vanillin polymers for enhanced skin coloration (U.S. Pat. Nos. 5.705,145, 6,214,322, 7,780,954, and 7,935,331, each incorporated herein by reference in its entirety). However, there is still a need for improved formulations capable of effectively darkening skin color while preserving the original hue of the skin.

### SUMMARY OF THE INVENTION

In view of the forgoing, there is a demand for a long-lasting topical skincare composition that produces deeper, richer colors without changing the skin's starting hue.

Accordingly, it is one object of the present invention to provide novel topical skincare compositions that meet these criteria.

It is another object of the present disclosure to provide novel methods of adjusting skin color appearance of a subject by topically applying the topical skincare composition onto the skin of the subject.

These and other objects, which will become apparent during the following detailed description, have been achieved by the inventors' discovery that the combination of a reducing sugar, an alkyl sulfonate, and a carrier unexpectedly yields topical skincare compositions that promote long lasting, rich, intense (i.e., dark), and natural looking tan color by increasing the saturation and darkness while maintaining the original hue of the skin color after topical application. Further, when an aromatic sulfonate is included, the topical skincare composition provides a custom tan by shifting the hue in addition to darkening and saturating the skin color.

Thus, the present invention provides:

(1) A topical skincare composition, comprising:

(A) about 0.1 to 30 wt. % of a reducing sugar relative to a total weight of the topical skincare composition;

(B) an optionally substituted C₈₋₁₂ alkyl sulfonate; and

(C) a carrier,

wherein a weight ratio of the optionally substituted C₈₋₁₂ alkyl sulfonate (B) to the reducing sugar (A) ((B):(A)) is 1:20 to 20:1.

(2) The topical skincare composition of (1), wherein the optionally substituted C₈₋₁₂ is alkyl sulfonate (B) is an unsubstituted alkyl sulfonate.

(3) The topical skincare composition of (1) or (2), wherein the optionally substituted C₈₋₁₂ alkyl sulfonate (B) is an alkali metal alkyl sulfonate salt.

(4) The topical skincare composition of any one of (1) to (3), wherein the optionally substituted C₈₋₁₂ alkyl sulfonate (B) is sodium 1-octanesulfonate, sodium 1-decanesulfonate, or both.

(5) The topical skincare composition of any one of (1) to (4), wherein the optionally substituted C₈₋₁₂ alkyl sulfonate (B) is sodium 1-octanesulfonate.

(6) The topical skincare composition of any one of (1) to (5), wherein a weight ratio of the optionally substituted C₈₋₁₂ alkyl sultanate (B) to the reducing sugar (A) ((B):(A)) is 1:20 to 20:1.

(7) The topical skincare composition of any one of (1) to (6), wherein the carrier (C) comprises an aromatic alcohol.

(8) The topical skincare composition of any one of (1) to (7), wherein the carrier (C) comprises benzyl alcohol.

(9) The topical skincare composition of any one of (1) to (8), further comprising (D) an organic solvent.

(10) The topical skincare composition of (9), wherein the organic solvent (D) is at least one selected from the group consisting of 1,3-propanediol. 1,2-propanediol, and 1,3-butanediol.

(11) The topical skincare composition of any one of (1) to(10), which is substantially free of a cationic copolymer.

(12) The topical skincare composition of any one of (1) to (11), further comprising (E) an aromatic sulfonate, wherein the aromatic sulfonate (E) has a topological polar surface area (tPSA) of less than 100 Å².

(13) The topical skincare composition of (12), wherein the aromatic sulfonate (E) is an optionally substituted phenyl sulfonate, an optionally substituted naphthyl sulfonate, or both.

(14) The topical skincare composition of (12) or (13), wherein the aromatic sulfonate (E) is at least one selected from the group consisting of sodium 2-naphthalenesulfonate, sodium p-toluenesulfonate, and sodium cumenesulfonate.

(15) The topical skincare composition of any one of (12) to (14), wherein a weight ratio of the alkyl sulfonate (B) to the aromatic sulfonate (E) ((B);(E)) is 1:100 to 100:1.

(16) A method of darkening color and maintaining hue of the skin of a subject, the method comprising:

topically applying the topical skincare composition of any one of (1) to (15) onto the skin of the subject,

wherein the topical application reduces a lightness L* of the color by at least 10%, and changes a hue angle h° of the color by less than 2°, each compared to those prior to the topical application.

(17) The method of (16), wherein the topical skincare composition is topically applied to the subject 1 to 3 times daily for 1 to 7 consecutive days.

(18) A method of darkening color of the skin of a subject, the method comprising:

topically applying the topical skincare composition of any one of (1) to (15) onto the skin of the subject,

wherein the topical application reduces a lightness L* of the color by at least 10% compared to that prior to the topical application.

(19) A method of adjusting color saturation of the skin of a subject, the method comprising:

topically applying the topical skincare composition of any one of (1 ) to (15) onto the skin of the subject,

wherein the topical application increases a saturation C* of the color by at least 10% compared to that prior to the topical application.

(20) A collection of topical skincare products for retail sale, the collection comprising:

(a) a first topical skincare composition that comprises:

(A) about 0.1 to 30 wt. % of a reducing sugar relative to a total weight of the topical skincare composition;

(B) an optionally substituted C₈₋₁₂ alkyl sulfonate; and

(C) a carrier,

wherein a weight ratio of the optionally substituted C₈₋₁₂ alkyl sulfonate (B) to the reducing sugar (A) ((B):(A)) is 1:20 to 20:1, and

(b) a second topical skincare composition that comprises:

(A) about 0.1 to 30 wt. % of a reducing sugar relative to a total weight of the topical skincare composition;

(B) an optionally substituted C₈₋₁₂ alkyl sulfonate; and

(C) a carrier,

wherein a weight ratio of the optionally substituted C₈₋₁₂ alkyl sulfonate (B) to the reducing sugar (A) ((B):(A)) is 1:20 to 20:1,

wherein a content of the reducing sugar (A) present in the first topical skincare composition (a) is less than that of the reducing sugar (A) present in the second skincare composition (b), and wherein the contents are each relative to total weights of the first and second topical skincare compositions.

(21) The collection of (20), wherein the first topical skincare composition (a) and the second topical skincare composition (b) are separately packaged.

(22) The collection of (20) or (21), wherein the content of the reducing sugar (A) relative to a total weight of the first topical skincare composition (a) is less than 5%, and the content of the reducing sugar (A) relative to a total weight of the second topical skincare composition (b) is greater than 5%.

(23) A topical skincare composition, comprising:

(A) about 0.1 to 30 wt. % of a reducing sugar relative to a total weight of the topical skincare composition;

(B) an optionally substituted C₈₋₁₂ alkyl sulfonate; and

(C) a carrier,

wherein the reducing sugar (A) is dihydroxyacetone, erythrulose, or both,

wherein the optionally substituted C₈₋₁₂ alkyl sulfonate (B) is an unsubstituted alkyl sulfonate.

wherein the carrier (C) comprises an aromatic alcohol, and

wherein a weight ratio of the optionally substituted C₈₋₁₂ alkyl sulfonate (B) to the reducing sugar (A) ((B):(A)) is 1:20 to 20:1.

(24) The topical skincare composition of (23), further comprising (D) an organic solvent,

(25) The topical skincare composition of (23), which is substantially free of a cationic copolymer.

(26) The topical skincare composition of (23), further comprising (E) an aromatic sulfonate, wherein the aromatic sulfonate (E) has a topological polar surface area (tPSA) of less than 100 Å².

(27) A topical skincare composition, comprising:

(A) about 0.1 to 10 wt. % of a reducing sugar relative to a total weight of the topical skincare composition;

(B) about 0.1 to 10 wt. % an optionally substituted C₈₋₁₂ alkyl sulfonate relative to a total weight of the topical skincare composition; and

(C) about 0.1 to 5 wt. % a carrier relative to a total weight of the topical skincare composition,

wherein the reducing sugar (A) is dihydroxyacetone, erythrulose, or both,

wherein the optionally substituted C8-12 alkyl sulfonate (B) is an unsubstituted alkyl sulfonate,

wherein the carrier (C) comprises an aromatic alcohol, and

wherein a weight ratio of the optionally substituted C₈₋₁₂ alkyl sulfonate (B) to the reducing sugar (A) ((B):(A)) is 1:5 to 5:1.

(28) The topical skincare composition of (27), further comprising (D) an organic solvent,

(29) The topical skincare composition of (27), which is substantially free of a cationic copolymer.

(30) The topical skincare composition of (27), further comprising (E) an aromatic sulfonate, wherein the aromatic sulfonate (E) has a topological polar surface area (tPSA) of less than 100 Å².

The foregoing paragraphs have been provided by way of general introduction, and are not intended to limit the scope of the following claims. The described embodiments, together with further advantages, will be best understood by reference to the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete appreciation of the disclosure and many of the attendant advantages thereof will be readily obtained as the same becomes better understood by reference to the following detailed description when considered in connection with the accompanying drawings, wherein:

FIG. 1 is a bar graph illustrating changes in skin darkness 6 hours and 24 hours after applications of a control composition (A) having DHA alone, and a topical skincare (B) composition containing DHA and sodium 1-decanesulfonate (SDS), respectively.

FIG. 2 is a color calibrated image showing human skin after treatment with a control composition having DHA alone (“control”)(top circle) and with a topical skincare composition containing DHA and SDS (“SDS”)(bottom circle), where color calibration was performed using the CASMATCH method (Bear Medic Co.).

FIG. 3 is a bar graph showing changes in skin darkness after 3 daily applications (1 application per day for 3 consecutive days), and 7 daily applications (1 application per day for 7 consecutive days) of a control composition (A) having DHA alone, and a topical skincare composition (B) containing DHA and sodium 1-octanesulfonate (SOS), respectively, as well as 3 days regression after the 7 daily applications.

FIG. 4 is a bar graph showing changes in skin hue angle after 3 daily applications (1 application per day for 3 consecutive days), and 7 daily applications (1 application per day for 7 consecutive days) of a control composition (A) having DHA alone, and a topical skincare composition (B) containing DHA and SOS, respectively, as well as 3 days regression other the 7 daily applications.

FIG. 5 is a bar graph summarizing changes in skin darkness 4 hours. 24 hours, and 48 hours other applications of a control composition (A) having DHA alone, and topical skincare compositions each containing (B) DHA and SOS, (C) DHA and sodium 1-decanesulfonate (SDS), (D) DM and is sodium dodecylbenzene sulfonate (SDBS), and (E) DM and sodium lauryl sulfoacetate (SLSA), respectively.

FIG. 6A is a bar graph summarizing changes in skin darkness after applications of a control composition having DHA alone (DHA), a topical skincare composition containing DI-JA and SOS (+SOS), a control composition containing DHA and sodium 2-naphthalenesulfonate (NSA) (+NSA), and a topical skincare composition containing DHA, SOS, and NSA (+NSA/SOS), respectively.

FIG. 6B is a picture showing human skin after treatment with a topical skincare composition containing DHA and SOS.

FIG. 6C is a picture showing human skin after treatment with a control composition containing DHA and NSA.

FIG. 6D is a picture showing human skin after treatment with a topical skincare composition containing DHA, SOS, and NSA.

FIG. 7 is a bar graph showing relative α-helix/β-sheet index ratios of keratins upon exposure to aqueous solutions containing SOS, SDS, and sodium lauryl sulfate (SLS), respectively,

FIG. 8 is a portion of a chromaticity diagram portrayed by CIE L*C*h° and CIE L*a*b* color space models,

FIG. 9A is a graph summarizing different degrees of darkening (L*) induced by DHA in the presence of SOS, SDS, and sodium lauryl sulfate (SLS), respectively.

FIG. 9B depicts keratin structural change from α-helix to β-sheet.

FIG. 10A is a picture showing dry stratum corneum.

FIG. 10B is a picture showing stratum corneum upon exposure to deionized water.

FIG. 10C is a picture showing stratum corneum upon exposure to a control composition having DHA alone.

FIG. 10D is a picture showing stratum corneum upon exposure to a topical skincare composition containing DHA and SOS.

FIG. 10E is a picture showing stratum corneum upon exposure to a topical skincare composition containing DHA, SOS, and 5 wt. % 1,3-propanediol (PD).

FIG. 10F is a picture showing stratum corneum upon exposure to a topical skincare composition containing DHA, SOS, and 10 wt. % PD.

FIG. 10G is a bar graph summarizing different degrees of darkening (L*) induced by a control composition having DHA alone (DHA), a topical skincare composition containing DHA and SOS (DHA/SOS), a topical skincare composition containing DHA, SOS and 5 wt. % PD (+5% PD), and a topical skincare composition containing DHA, SOS, and 10 wt. % PD (+10%PD), respectively.

### DETAILED DESCRIPTION OF THE INVENTION

In the following description, it is understood that other embodiments may be utilized and structural and operational changes may be made without departure from the scope of the present embodiments disclosed herein.

### Definitions

As used herein, the words “a” and “an” and the like carry the meaning of “one or more”.

Within the description of this disclosure, where a numerical limit or range is stated, the endpoints are included. Also, all values and subranges within a numerical limit or range are specifically included as if explicitly written out.

When referencing topical skincare compositions, the phrase “substantially free”, unless otherwise specified, describes an amount of a particular component present in the topical skincare composition being less than about 1 wt. %, preferably less than about 0.5 wt. %, more preferably less than about 0.1 wt. %, even more preferably less than about 0.05 wt. %, yet even more preferably 0 wt. %, relative to a total weight of the topical skincare composition.

As used herein, the word “about” may be used when describing magnitude and/or position to indicate that the value and/or position described is within a reasonable expected range of values and/or positions. For example, a numeric value may have a value that is +/− 0.1% of the stated value (or range of values), +/− 1% of the stated value (or range of values), +/− 2% of the stated value (or range of values), +/− 5% of the stated value (or range of values), or +/− 10% of the stated value (or range of values).

As used herein, the terms “optional” or “optionally” means that the subsequently described event(s) can or cannot occur or the subsequently described component(s) may or may not be present (e.g., 0 wt. %).

As used herein, the term “substituted” refers to at least one hydrogen atom that is replaced with a non-hydrogen group, provided that normal valencies are maintained and that the substitution results in a stable compound.

The term “alkyl”, as used herein, unless otherwise specified, refers to a straight, branched, or cyclic, aliphatic fragment having at least 1, preferably at least 2, preferably at least 3, preferably at least 4 carbon atoms and up to 22, preferably up to 20, preferably up to 18, preferably up to 12, preferably up to 8 carbon atoms. Examples of alkylgroups include, but are not limited to, methyl, ethyl, n-propyl, isopropyl, n-butyl, isobutyl, t-butyl, n-pentyl, isopentyl, neopentyl, n-hexyl, isohexyl, n-heptyl, n-octyl, n-nonyl, n-decyl, n-t decyl, n-dodecyl (lauryl), n-tetradecyl (myristyl), n-pentadecyl, n-hexadecyl (cetyl), n-octadecyl (stearyl), n-eicosanyl, 3-methylpentyl, 2,2-dimethylbutyl, 2,3-dimethylbutyl, and the like, including guerbet-type alkyl groups (e.g., 2-methylpentyl, 2-ethylhexyl, 2-proylheptyl, 2-butyloctyl, 2-pentylnonyl, 2-hexyldecyl, 2-heptylundecyl, 2-octyldodecyl, 2-nonyltridecyl, 2-decyltetradecyl, and 2-undecylpentadecyl). Cycloalkyl is a type of cyclized alkyl group. Exemplary cycloalkyl groups include, but are not limited to, cyclopropyl, cyclobutyl, cyclopentyl, cyclohexyl, norbornyl, and adamantyl.

As used herein, the ten “aryl” refers to an aromatic group containing only carbon in the aromatic ring(s), such as phenyl, biphenyl, naphthyl, anthracenyl, and the like.

The term “arylalkyl”, as used herein, refers to a straight, branched, or cyclic alkyl moiety (as defined above) that is substituted by an aryl group (as defined above) which may itself be optionally substituted by an alkyl group, examples of which include, but are not limited to, benzyl, phenethyl, 3-phenylpropyl, 2-phenylpropyl, 1-pheitylpropyl, 4-phenylbutyl, 3-phenylbutyl, 2-phenylbutyl, 2-methylbenzl, 3-methylbenzyl, 4-methylbenzyl, 2,4-dimethylbenzyl, 2-(4-ethylphenyl)ethyl, 3-(3-propylphenyl)propyl, and the like.

The term “alloy”, as used herein, refers to a straight or branched alkyl group attached to an oxygen atom. Exemplary alkoxy groups include, but are not limited to, methoxy, ethoxy, propoxy, isopropoxy, butoxy, isobutoxy, secondary butoxy, tertiary butoxy, pentoxy, isopentoxy, hexyloxy, heptyloxy, octyloxy, nonyloxy, and decyloxy.

The term “alkoxycarbonyl” as used in this disclosure refers to an alkoxy group bound to a carbonyl group (i.e., >C═O).

The term “halogen”, as used herein, means fluoro, chloro, bromo and iodo.

As used herein, the term “alkenyl”, unless otherwise specified, refers to a linear, branched, or cyclic, aliphatic fragment having 2 to 22 carbon atoms, preferably 3 to 20 carbon atoms, preferably 4 to 18 carbon atoms, and which contains at least one site of unsaturation. Examples of alkenyl groups include, but are not limited to, vinyl, allyl, 1-propenyl, 2-propenyl, -butenyl, 2-butenyl, 3-butenyl,1-pentenyl, 2-pentenyl, 3-pentenyl, 4-pentenyl, 1.-hexenyl, 2-hexenyl, 3-hexenyl, 4-hexenyl, 5-hexenyl, oleyl, linoleyl, and the like, including cycloalkenyl groups such as cyclopentenyl, cyclohexenyl, cycloheptenyl, and the like.

As used herein, the term “skin” refers to the skin that exists in humans and other mammals. It is to be recognized that skin exists on many different body parts, and application of the topical skincare compositions herein is not limited to skin found on a specific body part. For example, the topical skincare compositions may be applied to any area of the skin intended for self-tanning, including the face, limbs, feet, neck, torso, and the like. Further, the topical skincare composition may be applied as a moisturizer to the whole body in a daily skin care routine.

Various topical skincare composition ingredients are listed throughout the present disclosure and are organized according to their primary or most desired function, benefit, or use. However, categorization of an ingredient under a particular function, benefit, or use is not meant to limit that ingredient to only that function, benefit, or use. For example, listing of benzyl alcohol as a carrier does not limit the usefulness of benzyl alcohol to only that of a carrier, since benzyl alcohol can also impart other beneficial attributes, such as acting as a preservative and/or a fragrance.

### Topical Skincare Composition

The present disclosure is directed to sunless tanning topical skincare compositions that reduce lightness, increase color saturation, and create customized hue of skin appearance. The topical skincare compositions are easy to apply and may be used to darken the color of the skin, preserve or adjust the original tone of the skin, and provide a rich and long lasting tan.

The topical skincare composition therefore contain components which enables tanning of the skin and components which facilitate delivery of tanning agents and allow the topical skincare compositions to be easily applied to decrease the lightness and maintain the hue of the skin. Such compositions generally include the following components: a tanning agent, which is preferably (A) a reducing sugar, (B) an alkyl sulfonate, (C) a carrier, and optionally (D) an organic solvent, (E) an aromatic sulfonate, (F) water, (G) a thickening agent, (H) a preservative, and (I) an acidulent. In preferred embodiments, all components are compatible with the reducing sugar (i.e., do not react or cause the reducing sugar to react) and are homogeneously dispersed or dissolved uniformly throughout the topical skincare composition. It has been surprisingly found that the addition of the alkyl sulfonate to the reducing sugar enhances the self-tanning effect of the composition. For example, the presence of alkyl sulfonate(s) in a composition containing reducing sugar maintains original hue angle) (h°) in addition to increasing darkness and saturation (C*) of the color of the treated skin.

The topical skincare composition may be in a form of a liquid, a solution, an emulsion, a lotion, a cream, a gel, a paste, a spray, a foam, or any other form that is suitable for topical application to the skin. Preferably, the topical skincare composition is in the form of a lotion, a cream, a gel, a spray, or a foam. More preferably, the topical skincare composition is in the form of a cream or a gel that can be evenly applied.

### Tanning Agent

To act as an effective self-tanner, the topical skincare composition herein includes a “tanning agent”, which is any colored molecule that is capable of staining the skin when it is brought into contact with the skin, or any non-colored molecule that is capable of reacting with and coloring the skin, in particular, a molecule capable of darkening the skin so that it resembles the darkening effect achieved by exposure of one's skin to solar radiation (i.e., a natural tan).

In preferred embodiments, the tanning agent is (A) a reducing sugar. Certain reducing sugars such as monosaccharides (e.g. dihydroxyacetone) react with amino acids naturally occurring on the skin surface and, by virtue of a Maillard reaction, form pigmented melanoidins that cause the skin to change color (Bobin et al. J. Soc. Cosmet. Chem, 35 pages 265-272,1984; Maillard L. C., C. R. Acad. Sci. 154, 66-68,1912 —each incorporated herein by reference in its entirety).

Different amino acids react with different reducing sugars differently to produce a variety of tones of coloration from yellow to brown. Any reducing sugar capable of reacting with amino acids found in skins (e.g., naturally occurring amino acids) to produce a darkened skin, can be employed as the tanning agent herein. The reducing sugar (A) may be a monosaccharide which is an aldose having 2 to 6 carbon atoms, preferably 3 to 5 carbon atoms, more preferably 3 to 4 carbon atoms, even more preferably 3 carbon atoms, a ketose having 3 to 6 carbon atoms, preferably 4 to 5 carbon atoms, more preferably 3 to 4 carbon atoms, including mixtures such aldoses and; or ketoses. Exemplary reducing sugars include, but are not limited to, dihydroxyacetone (DHA), erythrulose, glycolaldehyde, glyceraldehyde, meso-tartaric aldehyde, glucose, gulose, xylose, fructose, ribose, arabinose, allose, talose, altrose, idose, mannose, galactose, and erythrose. In preferred embodiments, the reducing sugar (A) is dihydroxyacetone, erythrulose, or both. Most preferably, the reducing sugar (A) is dihydroxyacetone. Dihydroxyacetone is available, for example, from EMD Millipore.

The amount of tanning agent present in the topical skincare composition may vary-depending on the skin coloration (e.g., lightness, hue angle, color saturation) desired and the quantity and nature of the other components. In some embodiments, the tanning agent is present in amounts of at least about 0.05 wt. %, preferably at least about 0.1 wt. %, preferably at least about 0.5 wt. %, preferably at least about 1 wt. %, more preferably at least about 1.5 wt. %, even more preferably at least about 1.75 wt. %, yet even more preferably at least about 2 wt. %, and up to about 10 wt. %, preferably up to about 8 wt. %, preferably up to about 6 wt. %, preferably up to about 4 wt. %, preferably up to about 3.5 wt. %, more preferably up to about 3 wt. %, even more preferably up to about 2.5 wt. %, yet even more preferably up to about 2.25 wt. %, based on a total weight of the topical skincare composition.

In preferred embodiments, the topical skincare compositions are substantially free of tanning agents besides reducing sugars, which includes being substantially free of, preferably completely free of (i.e., 0 wt. %) synthetic dyes and natural pigments which provide color. Alternatively, the topical skincare compositions may include other tanning agent such as synthetic dyes and/or natural pigments in amounts listed previously.

Examples of synthetic dyes which may be incorporated as a tanning agent include, but are not limited to acid dyes (e.g., Yellow No. 203 (D&C Yellow No. 10 color index (CI) given as CI 47005), Orange No. 205 (D&C Orange No. 4, CI 15510), Red No. 3 (Erythrosin B, CI 45430), Red No. 94 (Bengal rose, CI 45440), and Red No. 227 (D&C Red No. 33, CI 17200)); quinone-based dyes (e.g., anthraquinone, 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone, 1,4-bis(β,γ-dihydroxypropylamino)anthraquinone, lawsone, juglone, alizarin, purpurin, carminic acid, carmine, kermesic acid, spinulosin, Disperse Red 15, Solvent Violet 13, Disperse Violet 1, Disperse Violet 4, Disperse Blue 1, Disperse Violet 8, Disperse Blue 3, Disperse Red 11, Disperse Blue 7, Basic Blue 22, Disperse Violet 15, Basic Blue 99); azo-based dyes (e.g., 1,3-dimethyl-2-[[4-(dimethylamino)phenyl]azo]1H-imidazolium chloride, 1,3-dimethyl-24(4-aminophenyllazokl1H-imidazolium chloride, 1-methyl-4-[(methylphenylhydrazono)methyl]pyridinium methyl sulfate, Disperse Red 17, Basic Red 22, Basic Red 76, Basic Yellow 57, Basic Brown 16, Basic Brown 17, Disperse Black 9); and indoamine-based dyes (e.g., 2-β-hydroxyethylamino-5[bis(β-4′-hydroxyethyl) amino]anilino-1,4-benzoquinone, 2-β-hydroxyethylamino-5-(2-methoxy-4′-amino)anilino-1,4-benzoquinone, 3 -N-(2′-chloro-4′-hydroxy)phenylacetamino-6-methoxy-1,4-benzoquinoneimine, 3-N-(3′-chloro-4′-methylamino)phenylureido-6-methyl-1,4-benzoquinoneimine, 3-[4′-N-(ethylcarbamylmethyl)amino]phenylureido-6-methyl-1,4-benzoquinoneimine).

The topical skincare composition may include a natural pigment as a tanning agent. Non-limiting examples of natural dyes include caramels, beta-carotenes, beet root extracts, blue green algae, cocoa powder, walnut extracts, melanin, and curcumin.

### (B) Alkyl Sulfonate

The topical skincare composition of the present disclosure may include an alkyl sulfonate (B). Preferably, the alkyl sulfonate (B) has formula (I)

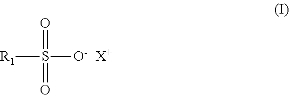

or a solvate thereof, a tautomer thereof, a stereoisomer thereof, or a mixture thereof, wherein (i) R₁ is an optionally substituted alkyl, or an optionally substituted arylalkyl, and (ii) X is a cation selected from the group consisting of hydrogen ion, ammonium ion, and an alkali metal ion.

The term “cation” means a positively charged ion including, but not limited to, hydrogen ion, ammonium ion (i.e., NH₄⁺), quaternary ammonium ion (e.g., tetraethyl ammonium, tetrabutyl ammonium), lithium ion, sodium ion, potassium ion, and silver ion. Preferably, the alkyl sulfonate (B) is an alkali metal alkyl sulfonate salt. In preferred embodiments, X is an alkali metal ion such as lithium ion, sodium ion, and potassium ion. Most preferably, X is sodium ion.

In some embodiments, R₁ is an optionally substituted C₁₋₂₂ alkyl, preferably an optionally substituted C₂₋₂₀ alkyl, preferably an optionally substituted C₃₋₁₈ alkyl, preferably an optionally substituted C₄₋₁₆ alkyl, preferably an optionally substituted C₅₋₁₄ alkyl, preferably an optionally substituted C₆₋₁₂ alkyl., preferably an optionally substituted C₇₋₁₀ alkyl, preferably an optionally substituted C₈₋₉ alkyl. The carbon counts described herein refers to a number of carbon atoms of the alkyl group of R₁ which excludes the carbon atoms of optionally present substituents.

In preferred embodiments, R₁ is an unsubstituted alkyl, preferably a linear alkyl, preferably a linear C₁₋₁₈ alkyl, preferably a linear C₂₋₁₆ alkyl, preferably a linear C₃₋₁₄ alkyl, preferably a linear C₄₋₁₂ alkyl, preferably a linear C₅₋₁₀ alkyl, preferably a linear C₆₋₉ alkyl, preferably a linear C₇₋₈ alkyl. Exemplary linear alkyls include, but are not limited to, methyl, ethyl, n-propyl, n-butyl, n-pentyl, n-hexyl, n-heptyl, n-octyl, n-nonyl, n-decyl, n-undecyl, n-dodecyl, n-tetradecyl, n-pentadecyl, n-hexadecyl, n-octadecyl, and n-eicosanyl. Alternatively, R₁ is a branched or cyclized alkyl, such as isopropyl, sec-butyl, isobutyl, isobutyl, tort-butyl, isopentyl, neopentyl, isohexyl, 2-methylpentyl, 3-methylpentyl, 2-ethylhexyl, 2-proylheptyl, 2-butyloctyl, 2-pentylnonyl, 2-hexyldecyl, 2-heptylundecyl, 2-octyldodecyl, 2-nonyltridecyl, 2-decyltetradecyl, 2-undecylpentadecyl, and cyclohexyl. In some embodiments, R₁ is an alkyl substituted with at least one substituent such as an alkoxy, an alkoxycarbonyl, carboxy, an amino, hydroxy, thiol, and a halogen. Most preferably, R₁ is n-octyl or n-decyl.

Disadvantages associated with using common reducing sugars (e.g., dihydroxyacetone, erythrulose) alone as tanning agents include insufficient and rapidly fading coloration, unnatural orange undertone, and uneven coverage. It has been unexpectedly discovered that a combination of a reducing sugar (A) (e.g., dihydroxyacetone) and an alkyl sulfonate (B), as will become dear, provides an intense, long-lasting artificial tan that preserves the original hue of the treated skin.

Exemplary alkyl sulfonates include, but are not limited to, unsubstituted alkyl sulfonates such as potassium methanesulfonate, potassium trifluoromethanesulfonate, sodium ethanesulfonate, sodium 1 -propanesulfonate, sodium 1-butanesulfonate, 1-pentanesulfonic acid sodium salt, sodium 1-hexanesulfonate, sodium 1-heptanesulforiate, sodium 1-octanesulfonate, sodium 1-nonanesulforiate, sodium 1-decanesulfonate, sodium 1-undecanesulfonate, sodium 1-dodecanesulfonate, sodium 1-tetradecanesulfonate, 1-pentadecanesulfonic acid sodium salt, 1-hexadecanesulfonic acid sodium salt, sodium 1-octadecanesulfonate, and sodium cyclohexanesulfonate substituted alkyl sulfonates such as sodium 2-bromoethanesulfonate, sodium 2-chioroethanesulfonate, 2-hydroxyethanesulfonic acid sodium salt, 2-hydroxyethanesulfonic acid ammonium salt, 3-hvdroxy-l-propanesulfonic acid sodium salt. 3-bromopropanesulfonic acid sodium salt, 3-chloro-2-hydroxypropanesulfonic acid sodium salt, sodium 3-mercapto-1-propanesulfonate, sodium 3-(N-ethyl-3-methylanilino)propanesulfonate, N-ethyl-N-(3-sulfopropyl)-m-anisidine sodium salt, N-ethyl-N-(2-hydroxy-3-sulfopropyl)-3,5-dimethoxyaniline sodium salt, 3-(N,N-dimethyltetradecylammonio)propanesulfonate, 3-(N,N-dimnethyloctadecylammonio)propanesulfonate, dioctyl sulfosuccinate sodium salt, and dicyclobexyl sulfosuccinate sodium salt, and mixtures thereof.

In some embodiments, alkenyl sulfonates (i.e., where R₁ of formula (I) is an alkenyl group instead) such as 1-octene-1-sulfonic acid sodium salt, 2-octene-1-sulfonic acid sodium salt, sodium 1-bydroxy-3,7-dimethyl-6-octene-1-sulfonate, 1-decene-1-sulfonic acid sodium salt, 1 -dodecene-1-sulfonic acid sodium salt, 2-dodecene-1-sulfonic acid sodium salt, sodium 1-tetradecene-1 -sulfonate, and sodium 1-hexadecene-1-sulfonate may be used in addition to, or in lieu of the aforementioned alkyl sulfonates.

In preferred embodiments, the alkyl sulfonate (B) is sodium 1-octanesulfonate, sodium 1-decanesulfonate, or both. More preferably, the alkyl sulfonate (B) is sodium 1-octanesulfonate.

The stratum corneum is the outermost layer of the skin epidermis containing corneocytes with keratin filaments embedded in a structurally organized water-lipid matrix, Keratin swelling may occur when certain chemicals such as harsh surfactants penetrate the stratum corneum layer and interact with keratins, causing disruption of the secondary and tertiary structures of keratins and excessive water diffusion. This process also depletes natural moisturizing components inherent to corneocytes such as amino acids and lipids. As a result, excessive keratin swelling may cause dryness, redness, itchiness, and irritation of the skin.

As used herein, relative keratin swellability of a compound is determined by comparing the degree of swelling of stratum corneum exposed to an aqueous solution containing 2% (w/w %) of the compound relative to that of the stratum corneum exposed to water. For example, the relative keratin swellability may be calculated by the following formula (A):

$$Relative Keratin Swellability , {S}_{relative} = \frac{Hsolution - Hdry}{Hwater - Hdry} \qquad ( A )$$

where Hₛₒₗᵤₜᵢₒₙ is e height of the stratum corneum when exposed to a test solution containing 2% (w/w %) of a test compound in deionized water, H_{dry}, is the height of the stratum corneum before adding a test solution or deionized water, and H_{water} is the height of the stratum corneum in deionized water. The height of stratum corneum in each sample is measured by taking a digital picture of the samples in a tube rack, uploading the image to ImageJ (NIH) software, and then utilizing its image analysis capabilities to measure the distance (see Example section for experimental details on determination of relative keratin swellability).

Alkyl sulfonates (B) applicable to the present disclosure may have a relative keratin swellability of less than 1.28 at a concentration of 2 wt. %, for example, from about 0.6, preferably from about 0.62, preferably from about 0.64, preferably from about 0.66, preferably from about 0.68, more preferably from about 0.70, even more preferably from about 0.72, yet even more preferably from about 0.74, and up to about 1.2, preferably up to about 1.15, preferably up to 1.1, preferably up to 1.05, preferably up to 1.0, preferably up to 0.95, preferably up to 0.9, preferably up to 0.85, more preferably up to 0.8, even more preferably up to 0.78, yet even more preferably up to 0.76.

Keratins are categorized into α-helix keratin and β-sheet keratin according to their secondary structures. Similar to other α-helix proteins, the α-helix keratin adopts a stable, coiled-coil structure. Compared to the α-helix keratin, the β-sheet keratin has a larger number of exposed side-chains (e.g., disulfide bonds, hydrogen bonds) Which facilitate intercalation of water molecules between the sheets. Thus, β-sheet keratin tends to swell more readily than α-helix keratin. The α-helix/β-sheet ratio of keratin may serve as an indicator of keratin swellability. Compounds generating a greater α-helix/β-sheet ratio of keratin may have reduced tendency to cause keratin swelling. Methods of calculating the α-helix/β-sheet ratio of keratin are known by those of ordinary skill in the art. For example, the ratio may be determined using Raman spectroscopy (e.g., confocal Raman microscopy). IR spectroscopy (e.g., ATR/FT-IR), NMR spectroscopy, and wide-angle X-ray scattering.

As used herein, relative α-helix/p-sheet index ratio of a compound is determined by comparing the α-helixβ-sheet index of stratum corneum exposed to an aqueous solution containing 2% (w/w %) of the compound relative to that of the stratum corneum exposed to water (see Example section for experimental details on determination of relative α-helix/β-sheet index ratio).

Alkyl sulfonates (B) applicable to the present disclosure have a relative α-helix/β-sheet index ratio of greater than 0.86 at a concentration of 2 wt. %, for example, from 0.87, preferably from 0.88, preferably from 0.89, preferably from 0.90, preferably from 0.91, preferably from 0.92, more preferably from 0.93, even more preferably from 0.93, and up to 0.99, preferably up to 0.98, preferably up to 0.97, more preferably up to 0.96, even more preferably up to 0.95.

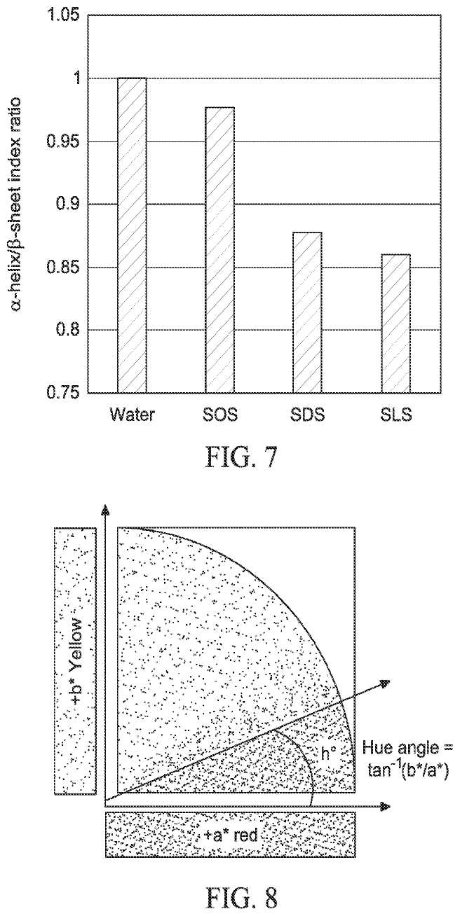

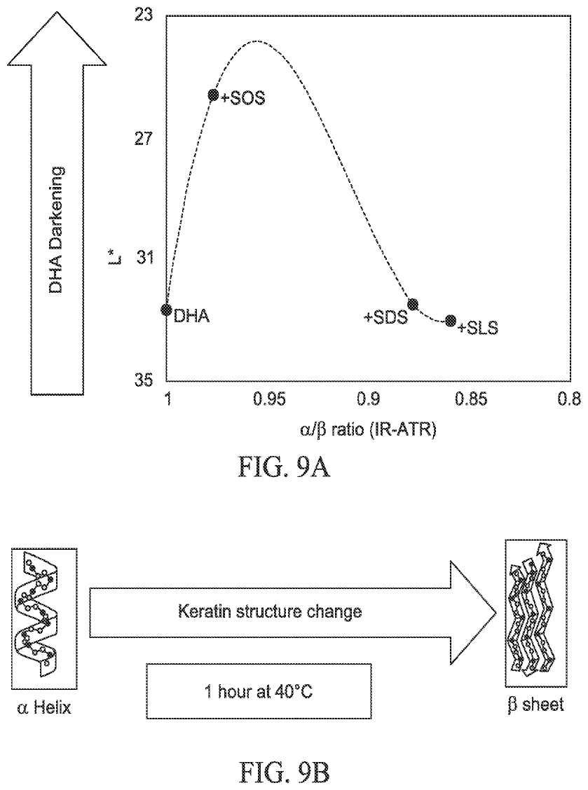

As shown in FIG. 7, exposure of keratin to sodium 1-octanesulfonate (SOS) leads to a smaller degree of phase transition from α-helix keratin to β-sheet keratin than exposure to 1-decanesulfonate (SDS), while sodium lauryl sulfate (SLS) induces the largest degree of such phase transition among the three. Keratins having a smaller α-helix/β-sheet ratio tend to have larger accessible amounts of lysine, which is one of the amino acids in skin that reacts most readily with tanning agents (e.g., dihydroxyacetone) via Maillard reaction. However, a smaller α-helix/β-sheet ratio may allow more water inside the keratin protein and cause swelling, which can hinder the Maillard reaction. Accordingly, compounds that can balance between swelling and structural change (e.g., SOS) may assist tanning agents in achieving effective color darkening by freeing up enough lysine residues but not allowing excessive water into the keratin protein. FIG. 9A shows that increasing the relative α-helix/β-sheet index ratio of a salt from about 0.86 (i.e., SLS) to about 0.97 (i.e., SOS) increases the skin darkening induced by DHA, while further increasing the relative α-helix/β-sheet index ratio beyond 0.97 may decrease the skin darkening induced by DHA,

In some embodiments, the alkyl sulfonate (B) is present in amounts of at least about 0.05 wt. %, preferably at least about 0.1 wt. %, preferably at least about 0.5 wt. %, preferably at least about 1 wt. %, more preferably at least about 1.5 wt. %, even more preferably at least about 2 wt. %, yet even more preferably at least about 2.5 wt. %, and up to about 10 wt. %, preferably up to about 8 wt. %, preferably up to about 6 wt. %, preferably up to about 5 wt. %, preferably up to about 4 wt. %, more preferably up to about 3.5 wt. %, even more preferably up to about 3 wt. %, yet even more preferably up to about 2.75 wt. %, based on a total weight of the topical skincare composition.

### (C) Carrier

The topical skincare compositions of the present disclosure may include a carrier (C), which is a material capable of enhancing uniform delivery and penetration of the tanning agent (and other components of the topical skincare composition) into the skin so that a deeper, richer, and longer-lasting tan can be achieved.

Examples of carriers (C) suitable for use herein include, but are not limited to, benzyl alcohol, 2-phenylethyl alcohol, phenoxyethanol, methanol, ethanol, n-propanol, iso-propanol, n-butanol, see-butanol, iso-butanol, tert-butanol, hexanol, n-octanol, 2-octanol, 2-ethyl hexonal, diethylene glycol monoethyl ether, diethylene glycol monobutyl ether, and mixtures thereof.

In some embodiments, the carrier (C) comprises an aromatic alcohol. Because of the presence of a polar end (hydroxy group) and a ion-polar end (phenyl group), aromatic alcohols (e.g., benzyl alcohol, 2-phenylethyl alcohol, phenoxyethanol) may enable a more effective transdermal delivery of the tanning agent (and other components of the topical skincare composition) than other short chain alkyl alcohols, such as ethanol and n-butanol. Furthermore, benzyl alcohol is particularly advantageous because of its moderate water solubility, aromatic odor, antimicrobial properties, and low toxicity. In preferred embodiments, the carrier (C) used herein comprises benzyl alcohol, 2-phenylethyl alcohol, 1-phenylethanol, phenoxyethanol, or mixtures thereof. Most preferably, the carrier (C) comprises, or consists essentially of benzyl alcohol (available from Emerald Kalama Chemical).

In some embodiments, the amount of carrier (C) present in the topical skincare composition is from about 0.1 wt. %, preferably from about 0.5 wt. %, preferably from about 1 wt. %, preferably from about 1.5 wt. %, preferably from about 2 wt. %, and up to about 5 wt. %, preferably up to about 4 wt. %, preferably up to about 3 wt. %. preferably up to about 2.5 wt. %, based on a total weight of the topical skincare composition.

The weight ratios among the reducing sugar (A) (e.g., dihydroxyacetone), the alkyl sulfonate (B) (e.g., sodium1-octanesulfonate, sodium 1-decanesulfonate), and the carrier (C) (e.g., benzyl alcohol) may be varied depending on tanning color shade (e.g., darkness, hue angle, color saturation). However, typically, a weight ratio of the alkyl sulfonate (B) to the reducing sugar (A) ((B):(A)) is from 1:20, preferably from 1:15, preferably from 1:10, preferably from 1:8, preferably from 1:6, preferably from 1:5, preferably from 1:4, preferably from 1:3, more preferably from 1:2, even more preferably from 2:3, yet even more preferably is from 1:1, and up to 20:1, preferably up to 15:1, preferably up to 10:1, preferably up to 8:1, preferably up to 6:1, preferably up to 5:1, preferably up to 4:1, preferably up to 3:1, more preferably up to 2:1, even more preferably up to 3:2, yet even more preferably up to 5:4.

In some embodiments, a weight ratio of the alkyl sulfonate (B) to the carrier (C) ((B):(C)) is from 1:4, preferably from 2:7, preferably from 1:3, preferably from 2:5, more preferably from 1:2, even more preferably from 2:3, yet even more preferably from 1:1, and up to 4:1, preferably up to 7:2, preferably up to 3:1, preferably up to 5:2, more preferably up to 2:1., even more preferably up to 3:2, yet even more preferably up to 5:4.

(D) Organic solvent

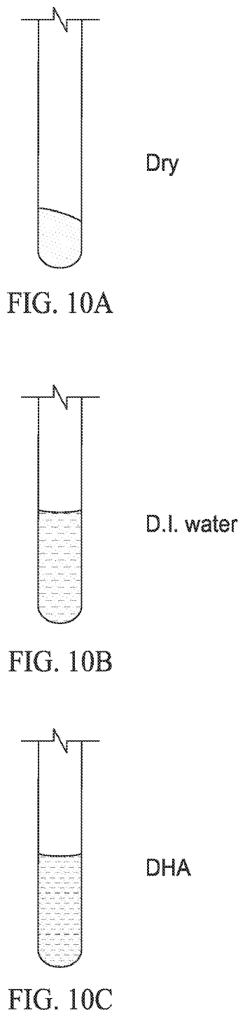

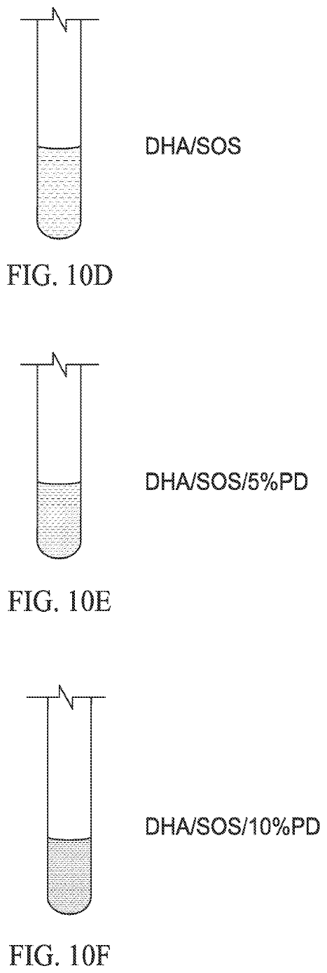

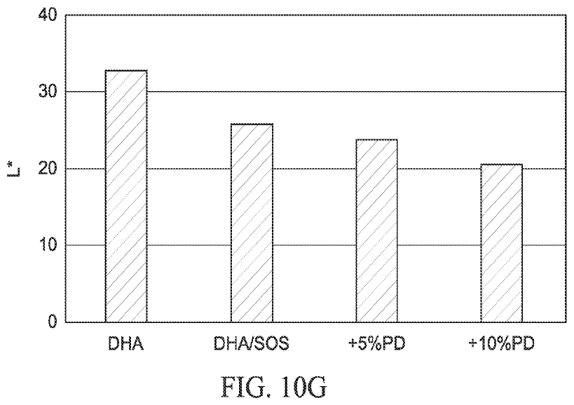

The topical skincare composition may optionally include an organic solvent (D), which is structurally different from the carder (C), The organic solvent (D) may aid solubilization of components not sufficiently soluble in the aforementioned carrier (C), adjust the surface property of the topical skincare composition for enhanced workability, viscosity, and/or ease of handling, or to generally provide a medium that is suitable for self-tanning operations. Examples of organic solvents useful for the present disclosure include, but are not limited to, polyols, for example, ethylene glycol, propylene glycol (e.g., 1,3-propanediol. 1,2-propanediol), butylene glycol (e.g., 1,3-butanediol, 1,2-butanediol, 1,4-butanediol, 2,3-butanediol), hexylene glycol, isoprene glycol, diethylene glycol, dipropylene glycol, glycerin, polyol ethers, for example, ethylene glycol monomethyl ether, ethylene glycol monoethyl ether, propylene glycol monomethyl ether, diethylene glycol monomethyl ether, diethylene glycol monoethyl ether), and a C₁ to C₄ lower alkanol, for example, methanol, ethanol, isopropanol, butanol, as well as mixtures thereof. In some embodiments, the organic solvent (D) comprises a polyol which is at least one selected from the group consisting of 1,3-propanediol, 1,2-propanediol, ethylene glycol, glycerin, and 1,3-butanediol (available from OXEA). In preferred embodiments, the organic solvent (D) is 1,3-propanediol. When present, polyols (e.g., 1,3-propanediol) may also enhance the Maillard reaction by reducing the amount of water that enters the keratin protein (see FIGS. 10A-G).

Alternatively, a mixture of polyols is used as the organic solvent (D), for example a mixture of a first polyol and a second polyol which is different from the first polyol. In some embodiments, the first polyol is 1.,3-propanediol. In some embodiments, the second polyol is glycerin. When a mixture of the first polyol 1,3-propanediol) and the second polyol (e.g., glycerin) is used, a weight ratio of the first polyol to the second polyol may be typically from 1:6, preferably from 1:5, preferably from 1:4, preferably from 1:3, more preferably from 1:2, more preferably from 1:1, even more preferably from 3:2, yet even more preferably from 2:1, d up to 10:1, preferably up to 8:1, preferably up to 6:1. more preferably up to 5:1, even more p p to 4:1, yet even more preferably up to 3:1.

When present, the organic solvent (D) may be included in the topical skincare compositions in an amount ranging from about 0.2 wt. %, preferably from about 0.5 wt. %, more preferably from about 1 wt: , preferably from about 2 wt. %, more preferably from about 3 wt. %, even more preferably from about 4 wt %, yet even more preferably from about 5 wt. %, and up to about 20 wt. %, preferably up to about 15 wt. %, preferably up to about 12 wt. %, more preferably up to about 10 wt. %, even more preferably up to about 8 wt. %, yet even more preferably up to about 6 wt. %, based on a total weight of the topical skincare composition.

A weight ratio of the carrier (C) (e.g., benzyl alcohol) to the organic solvent (D) 1,3-propanediol, ethylene glycol, glycerin) may range from 1:200, preferably from 1:150, preferably from 1:100. preferably from 1:50, preferably from 1:25, preferably from 1:20, preferably from 1:10, more preferably from 1:8, even more preferably from 1:6, yet even more preferably from 1:5, and up to 25:1, preferably up to 20:1, preferably up to 15:1, preferably up to 10:1. preferably up to 5:1, preferably up to 3:1. preferably up to 2:1, preferably up to 1:1, more preferably yap to 1:2, even more preferably up to 1:3, yet even more preferably up to 1:4.

### (E) Aromatic Sulfonate

The topical skincare composition of the present disclosure may optionally include an aromatic sulfonate (E). Preferably, the aromatic sulfonate (E) has formula (II)

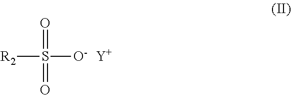

or a solvate thereof, a tautomer thereof, a stereoisomer thereof, or a mixture thereof, wherein (i) R₂ is an optionally substituted aryl or an optionally substituted heteroaryl, and (ii) Y is a cation selected from the group consisting of hydrogen ion, ammonium ion, and an alkali metal ion.

The cation Y may be hydrogen ion, ammonium ion (i.e., NH₄⁺), quaternary ammonium ion (e.g., tetraethyl ammonium, tetrabutyl ammonium), lithium ion, sodium ion, potassium ion, and silver ion. Preferably, the aromatic sulfonate (E) is an alkali metal aromatic sulfonate salt. In preferred embodiments, Y is an alkali metal ion such as lithium ion, sodium ion, and potassium ion. Most preferably, Y is sodium ion.

The aromatic sulfonate (E) may be an optionally substituted phenyl sulfonate, an optionally substituted naphthyl sulfonate, or both. In preferred embodiments, R₂ is an optionally substituted aryl. In some embodiments, R₂ is an optionally substituted phenyl. In some embodiments, R₂ is an optionally substituted naphthyl. R₂ may be substituted with at least one substituent such as an optionally substituted alkyl, an alkoxy, an alkoxycarbonyl, carboxy, an amino, hydroxy, thiol, halogen, cyano, and nitro. Alternatively, R₂ is unsubstituted.

In some embodiments, R₂ is substituted with at least one linear, branched, or cyclic alkyl group having at least 1, preferably at least 2, preferably at least 3, preferably at least 4, preferably at least 5 carbon atoms and up to 14, preferably up to 12, preferably up to 10, preferably up to 8, preferably up to 6 carbon atoms. In preferred embodiments, R₂ is substituted with at least one linear alkyl, such as methyl, ethyl, n-propyl, n-butyl, n-pentyl, n-hexyl, n-heptyl, n-octyl, n-decyl, and n-dodecyl. In preferred embodiments, R₂ is substituted with at least one branched alkyl, such as isopropyl, sec-butyl, isobutyl, isobutyl, tent-butyl, isopentyl, neopentyl, and isohexyl. Most preferably, R₂ is substituted with methyl or isopropyl.

Exemplary aromatic sulfonates include, but are not limited to, phenyl sulfonates such as sodium benzenesulfonate, sodium p-toluenesulfonate, sodium cumenesulfonate (i.e., sodium 4-isopropylbenzenesulfonate), sodium 2,3-dimethylbenzenesulfonate, sodium 2,5-dimethylbenzenesulfonate, 2,4,6-trimethylbenzenesulfonate, sodium i-1-ethylbenzenesulfonate, sodium 4-propylbenzenesulfonate, sodium 4-tent-butylbenzenesulfonate, sodium 4-chlorobenzenesulfonate, sodium 4-bromobenzenesulfonate, sodium 4-hydroxybenzenesulfonate, sodium dodecylbenzenesulfonate, sodium 3-sulfobenzoate, potassium 4-sulfobenzoate, 4-amino-3,5-dibromobenzenesulfonic acid sodium salt, 4-octylbenzenesulfonic acid sodium salt, and 4-dodecylbenzenesulfonic acid sodium salt; naphthyl sulfonates such as sodium i-naphthalenesulfonate, sodium 2-naphthalenesulfonate, sodium 2-methyl-l-naphthalenesulfonate, sodium 4-methyl-1-naphthalenesulfonate, sodium 2-butyl-1-naphthalenesulfonate, sodium 4-hydroxy-1-naphthalenesulfonate, sodium 4-amino-1 -naphthalenesulfonate, sodium 6-hydroxy-2-naphthalenesulfonate, and sodium 5-amino-1-naphthalenesulfonate, and mixtures thereof.

In some embodiments, polymers or oligomers containing the aromatic sulfonate of formula (II) as a repeating unit, for example poly(sodium 4-styrenesulfonate), poly(4-styrenesulfonic acid) ammonium salt, poly(4-styrenesulfonic acid), poly(sodium 2-styrenestilfonate), and sodium polyanetholesulfonate, may be used in lieu of, or in addition to the aforementioned aromatic sulfonates.

In preferred embodiments, the aromatic sulfonate (E) is at least one selected from the group consisting of sodium 2-naphthalenesulfonate, sodium p-toluenesulfonate, sodium cumenesulfonate, 4-dodecylbenzenesulfonic acid sodium salt, and poly(sodium 4-styrenesulfonate, more preferably at least one selected from the group consisting of sodium 2-naphthalenesulfonate (available from Sugai Chemical), sodium p-toluenesulfonate, and sodium cumenesulfonate.

As used herein, topological polar surface area (tPSA) of a molecule is defined as the sum of surface contributions of polar atoms (e.g., oxygen and nitrogen atoms, as well as bonded hydrogen atoms) in the molecule. Methods of calculating tPSA are known by those of ordinary skill in the a (see, e.g., Erti, P., et al., “Fast calculation of molecular polar surface area as a sum of fragment based contributions and its application to the prediction of drug transport properties”, J. Med. Chem. 2000, 43, 3714-3717, hereby incorporated by reference in its entirety). For example, tPSA can be determined using a desktop computer and commercially available chemical graphic software, such as ChemAxon-Marvinview and ChemDraw. Alternatively, tPSA can be found on many chemical databases, such as SciFinder.

Aromatic sulfonates (E) useful in the present disclosure have a topological polar surface area (tPSA) of less than 100 Å², for example, from about 20 Å², preferably from about 30 Å², more preferably from about 40 Å², even more preferably from about 50 Å², and up to about 95 Å², preferably up to about 90 Å², preferably up to about 80 Å², preferably up to about 70 Å², more preferably up to about 65 Å², even more preferably up to about 60 Å².

As used herein, hydrogen-bond donor sites are functionalities having hydrogen atoms which are readily coordinated with an electronegative atom such as oxygen, nitrogen, and sulfur. Exemplary hydrogen-bond donor sites include hydroxy, carboxylic acid, thiol, sulfonic acid, primary amines, secondary amines, and N-H functionalities in amides. In some embodiments, the aromatic sulfonate (E) has up to 2 hydrogen-bond donor sites, preferably up to 1 hydrogen-bond donor site, more preferably 0 hydrogen-bond donor sites.

While not wishing to be bound b theory, aromatic sulfonates (E) 4th a lower tPSA value and/or a smaller number of hydrogen-bond donors may have better skin permeability and higher dermal absorption, which can be advantageous for self-tanning skincare formulations.

When present, the aromatic sulfonate(E) may be included in the topical skincare composition in amounts of at least about 0.05 wt. %, preferably at least about 0.1 wt. %, preferably at least about 0.5 wt. %, preferably at least about 1 wt. %, more preferably at least about 1,5 wt. % even more preferably at least about 2 wt. %, yet even more preferably at least about 2.5 wt. %, and up to about 10 wt. %. preferably up to about 8 wt. %, preferably up to about 6 wt. %, preferably up to about 5 wt. %, preferably up to about 4 wt. %, more preferably up to about 3.5 wt. %, even more preferably up to about 3 wt. %, yet even more preferably up to about 2.75 wt. %, based on a total weight of the topical skincare composition.

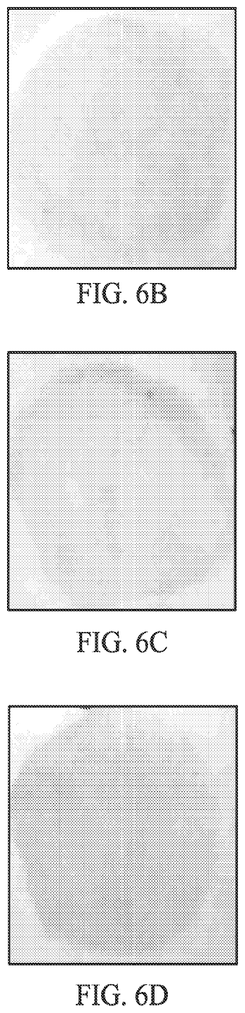

Without being bound by theory, it is believed that the aromatic sulfonate (E) herein promotes advanced Maillard reaction by aiding polymerization of melanoidins with a higher degree of polymerization and/or conjugation, thereby providing intensely-colored and fade resistant tans. It has been unexpectedly discovered that addition of an aromatic sulfonate (E) to a topical skincare composition containing a reducing sugar (A) (e.g., dihydroxyacetone) and an alkyl sulfonate (B) synergistically promotes the tanning performance of the composition by providing an intense, long-lasting artificial tan with customizable skin hue modification. Specifically, using a combination of the reducing sugar (A), the alkyl sulfonate (B), and the aromatic sulfonate (E) creates a significantly darker tan than using a mixture of the reducing sugar (A) and the alkyl sulfonate (B) alone, or a mixture of the reducing sugar (A) and the aromatic sulfonate (E) alone (see FIGS. 6A-D).

Custom shades of tan may be achieved by varying the weight ratio of the alkyl sulfonate (B) to the aromatic sulfonate (E). These variations yield topical skincare compositions with adjustable degrees of darkness and hue upon application. In some embodiments, a weight ratio of the alkyl sulfonate (B) to the aromatic sulfonate (E) ((B):(E)) is from 1:100, preferably from 1:90, preferably from 1:80, preferably from 1:70, preferably from 1:60, preferably from 1:50, preferably from 1:40, preferably from 1:30, preferably from 1:20, preferably from 1:10, more preferably from 1:8, preferably from 1:6, preferably from 1:5. preferably from 1:4, preferably from 1:3, even more preferably from 1:2, yet even more preferably from 1:1, and up to 100:1, preferably up to 90:1, preferably up to 80:1, preferably up to 70:1, preferably up to 60:1, preferably up to 50:1, preferably up to 40:1, preferably up to 30:1, more preferably up to 20:1, preferably up to 10:1, preferably up to 8:1, preferably up to 6:1, preferably up to 4:1, more preferably up to 3:1, even more preferably up to 2:1, yet even more preferably up to 3:2.

### (F) Water

In some embodiments, the topical skincare composition of the present disclosure is an aqueous composition or an oil-in-water (o/w) emulsion where the continuous phase is aqueous. Therefore, in preferred embodiments, the topical skincare composition further includes water (F) in amounts of at least about 10 wt. %, preferably at least about 20 wt. %, preferably at least about 30 wt. %, preferably at least about 40 wt. %, more preferably at least about 50 wt. %, even more preferably at least about 60 wt. %, yet even more preferably at least about 70 wt. %, and up to about 95 wt. %, preferably up to about 90 wt. %, more preferably up to about 85 wt. %, even more preferably up to about 80 wt. %, based on a total weight of the topical skincare composition.

### (G) Thickening Agent

The topical skincare composition may optionally include a thickening agent (G) that may improve stability of the composition as well as yield a consistency that is soothing to the skin upon application.

The thickening agent (G) may comprise a copolymer which contains, as a structural unit, at least one selected from the group consisting of a hydroxyalkyl acrylate, an acrylic acid salt, acrylamide, and an acryloyldialkyl taurate. Examples of thickening copolymers include a copolymer of hydroxyethyl acrylate and sodium acryloyldimethyl taurate, a copolymer of acrylate and sodium acryloyldimethyl taurate, a copolymer of acrylamide and acrylate, and a copolymer of acrylic acid, acrylamide, acrylate, and sodium acryloyldimethyl taurate. Preferably, the thickening agent (G) contains an anionic (co)polymer.

These thickening copolymers are commercially available, for example from SEPPIC, France. Exemplary thickening agents containing a copolymer of hydroxyethyl acrylate and acryloyldimethyl taurate include SEPINOV™ EMT 10 (hydroxyethyl acrylate/sodium acryloyldimethyl taurate copolymer), SIMULGEL™ NS (hydroxyethyl acrylate/sodium acryloyldimethyl taurate copolymer, squalane, polysorbate 60), SIMULGEL™ FL (hydroxyethyl acrylate/sodium acryloyldimethyl taurate copolymer, Isohexadecane, polysorbate 60), SEPIPLUS™ S (hydroxyethyl acrylate/sodium acryloyldimethyl taurate copolymer, polyisobutene, PEG-7 trimethylolpropane coconut ether), and SIMULGEL™ INS 100 (hydroxyethyl acrylate/sodium acryloyldimethyl taurate copolymer, Isohexadecane, polysorbate 60). Exemplary thickening agents containing a copolymer of acrylate and acryloyldimethyl taurate include SIMULGELT™ EG (sodium acrylate/sodium acryloyldimethyl taurate copolymer, isohexadecane, polysorbate 80), SIMULGEL™ EPG (sodium acrylate/sodium acryloyldimethyl taurate copolymer, polyisobutene, caprylyl capryl glucoside), arid SIMULGEL™ SMS 88 (sodium acrylate/acryloyldimethyl taurate/dimethyaciylamide cross-polymer, isohexadecane, polysorbate 60). An example of thickening agent containing a copolymer of acrylamide and acrylate include SEPIPLUS™ 265 (acrylamide/ammonium acrylate copolymer, polyisobutene, polysorbate 20). An example of thickening agent containing a copolymer of acrylic acid, acrylamide, acrylate, and acryloyldimethyl taurate includes SEPIPLUS™ 400 (polyacrylate-13, polyisobutene, polysorbate 20).

In some embodiments, the thickening agent (G) contains a copolymer of hydroxyethyl acrylate and a sodium acryloyldimethyl taurate. More preferably, the thickening agent is SEPIPLUS™ S (hydroxyethyl acrylate/sodium acryloyldimethyl taurate copolymer, polyisobutene, PEG-7 trimethylolpropane coconut ether).

Other thickening materials that may be used in addition to, or in lieu of the aforementioned thickening agents include carbomers (e.g., Carbomer 980), C₁₀₋₃₀ alkylacrylate cross-polymers (e.g., Pemulen TR-1, Pemulen TR-2 (acrylates/C₁₀₋₃₀ alkyacrylates cross-polymer)), SEPTGEL™ 305 (Polyacrylamide, C₁₃₋₁₄ isoparaffin, laureth-7), SIMULGEL™ A (ammonium polyacrylate, isohexadecane, PEG-40 castor oil), SIMULGEL™ 600 (acrylamide/sodium acryloyldimethyl taurate copolymer, isohexadecane, polysorbate 80), ARISTOFELX® AVC (ammonium acryloyldimethyltaurate/N-vinylpyrrolidone copolymer), ARISTOFIELX® AVS (sodium acryloyldimethyltaurate/N-vinylpyrrolidone copolymer), and modified cellulose polymers (e.g., hydroxyethyl cellulose, methyl cellulose).

A cationic copolymer, as used herein, unless otherwise specified, refers to a copolymer that incorporates a cationic monomer which is a methactyloylethyl tri(C₁-C₃ alkly) ammonium salt or an acryloylethyl tri(C₁-C₃ alkyl) ammonium salt (e.g., acryloylethyl trimethylammonium chloride) as one of the repeating units. Exemplary monomers that may be utilized in terming the cationic copolymer with the methacryloylethyl tri(C₁-C₃ alkly) ammonium salt or the acryloylethyl tri(C₁-C₃ alkyl) ammonium salt include acrylamide, methacrylamide, tris(hydroxymethyl)-acrylamidomethane, and those disclosed in U.S. Pat. No. 7,780,954—which is incorporated herein by reference in its entirety.

In preferred embodiments, the topical skincare compositions are substantially free of the cationic copolymer, which includes being substantially free of, preferably completely free of (i.e., 0 wt. %) the aforementioned cationic copolymer, such as acrylamide/acryloylethyl trimethylammonium chloride/tris(hydroxymethyl)-acrylamidomethane copolymer.

### (H) Preservative

The topical skincare composition may optionally further include a preservative (H), For example, the preservative may be selected to kill bacteria that might otherwise be sustained or multiplied in the composition, or to prevent degradation or chemical breakdown (e.g., oxidative degradation) of the composition. Preservatives suitable for use in cosmetic formulations are well-known to those of ordinary skill in the art. In this respect, the preservative chosen may be varied depending on the particular components present in the topical skincare composition. Illustrative of suitable preservatives include methylparaben, ethylparaben, propylparaben, EDTA or salts thereof (such as disodium EDTA), phenoxyethanol, DMDM hydantoin, benzyl alcohol, ethyldibromoglutaronitrile-phenoxyethanol/polyquatemium-7 (Euxyl K-400, Calgon), imidazolidinyl urea, diazolidinyl urea, benzalkonium chloride, benzethonium chloride, sodium benzoate, sorbic acid and the like, or combinations thereof.

Preferably, the preservative (H) is methylparaben and/or ethylparaben, most preferably a mixture of these preservatives. When present, the preservative (H) may be included herein in amounts of up to about 5 wt. %, preferably up to about 4 wt. %, preferably up to about 3 wt. %, preferably up to about 2 wt. %, preferably up to about 1 wt. %, preferably up to about 0.5 wt. %, for example from about 0.001 wt. % to about 3 wt. %, or 0.1 wt. % to about 1.5 wt. %, or 0,15 wt. % to about 1 wt. %, or 0,3 wt. % to about 045 wt. %, based on a total weight of the topical skincare composition.

### (I) Acidulant

The topical skincare compositions disclosed herein may be optionally formulated to include an acidulant (I) for adjusting the pH to be more acidic/less alkaline. Additionally, depending on the chemical structure, the acidulant (I) may act as a chelating agent and/or a buffering agent to neutralize minerals, enhance the activity of any preservatives present, and to stabilize active ingredients (e.g., the tanning agent).

The acidulant employed herein may be an inorganic acid or an organic acid, and specifically includes, but is not limited to, hydrochloric acid, orthophosphoric acid, sulfuric acid, carboxylic acids such as fumaric acid, acetic acid, and α-hydroxy acids such as tartaric acid citric acid, malic acid, lactic acid, and glycolic acid, as well as mixtures thereof. When the acidulant contains α-hydroxy acid functionality, the acidulant may also aid in exfoliating the skin and softening wrinkles. Preferably citric acid is used.

When present, the acidulant (I) may be included herein in amounts of up to about 5 wt. %, preferably up to about 4 wt. %, preferably up to about 3 wt. %, preferably up to about 2 wt. %, preferably up to about 1 wt. %, for example from about 0.001 wt. % to about 3 wt. %, or 0.02 wt. % to about 2 wt %, or 0.1 wt. % to about 1 wt %, or 0.2 wt. % to about 0.5 wt. %, based on a total weight of the topical skincare composition. The pH of the topical skincare composition may be varied, but is preferably less than 6.5, for example, at least 2, preferably at least 2.5, more preferably at least 3, even more preferably at least 3.5, and up to 6, preferably up to 5, more preferably up to 4.

### Other Optional Ingredients

Various optional ingredients frequently used in topical formulations such as fragrances, propellants, vehicles, adjuvants, anti-aging components, proteins, rheology control agents, dispersants, thickeners, film-forming agents, sequestering agents, cleansing agents, vitamins, botanicals, and sunscreen agents, as well as other classes of materials whose presence may be cosmetically, medicinally or otherwise desirable, can also optionally be included at their conventional art-established usage levels. For example, the topical skincare compositions of the present disclosure may be optionally formulated to include one or more fragrances known to those of ordinary skill in the cosmetics arts to impart a pleasant scent or to help mask any malodorous components that may be present in the topical skincare compositions.

In preferred embodiments, the topical skincare compositions are substantially free of such optional ingredients, however, when included, non-limiting examples which can be used include film-forming materials such as petrolatum, hydrolyzed wheat protein/wheat oligosaccharides (e.g., Cropeptide W by Croda Inc.), hydrolyzed corn protein, hydrolyzed wheat gluten, hydrolyzed yeast protein, hydrolyzed vegetable protein, hydrolyzed soy protein, hydrolyzed rice protein, and hydrolyzed potato protein; moisturizers such as glycereth-7-triacetate (Dermol GL-7-A_{;} Alto), glycerin, glycereth-5-lactate, and glycereth-7-diisononanoate; skin conditioning agents and emollients such as mineral oil, cetearyl alcohol, silicones, for example, dimethicone, cyclomethicone, phenyltrirnethicone, alkyl dimethicone, fluorinated silicones, esters of isononanoic acid, for example, ethylhexyl isononanoate butylene glycol diisononanoate, cetearyl isononanoate, and cetyl isononanoate, and polyethylene glycol derivatives of castor oil, for example, PEG-40 castor oil (Surfactol 365, available from Vertellus), PEG-45 castor oil, PEG-50 castor oil, PEG-60 castor oil, and PEG-100 castor oil; surfactants such as polyoxyalkylene ethers of a fatty alcohol, for example, laureth-3, ceteareth-6, ceteareth-11, ceteareth-15, ceteareth-16, ceteareth-17, ceteareth-18, ceteareth-20, ceteareth-23, ceteareth-25, ceteareth-27, ceteareth-28, ceteareth-30, isoceteth-20, laureth-9/myreth-9, and PPG-3 caprylyl ether, steareths (steareth-2, steareth-4, steareth-6, steareth-7, steareth-10, steareth-11, steareth-13, steareth-15, steareth-20), and polyethylene glycol esters, for example, PEG-14 laurate, PEG-1.5 laurate, PEG-20 laurate, PEG-32 laurate, PEG-75 laurate, PEG-150 laurate or other surfactants; and sunscreens or UV light absorbing compounds such as octyldimethyl PABA, benzophenone-4, DEA methoxycinnamate, 2-phenyl-benzimidazole-5-sulfonic acid, and triethanolamine salicylate.

In preferred embodiment, the topical skincare composition includes 1 to 3 wt. % of a reducing sugar (A) (e.g., dihydroxyacetone, erythrulose), 1 to 3 wt. % of an alkyl sulfonate (B) (e.g., sodium 1-octanesulfonate, sodium 1-decanesulfonate), 1 to 3 wt. % of a carrier (C) (e.g., benzyl alcohol), 2 to 8 wt. % of an organic solvent (D) (e.g., 1,3-propanediol, 1,2-propanediol, ethylene glycol, glycerin, 1,3-butanediol), and 70 to 82 wt. % water (F), each based on a total weight of the topical skincare composition, with the balance optionally including one or more of an aromatic sulfonate (E) (e.g., sodium 2-naphthalenesulfonate, sodium p-toluenesulfonate), a preservative (H) (e.g., a mixture of methylparaben and ethylparaben), an acidulent (I) (e.g., citric acid), and a thickening agent (G) (e.g., SEPIPLUS S (hydroxyethyl acrylate/sodium acryloyldimethyl taurate copolymer, polyisobutene, PEG-7 trimethylolpropane coconut ether).

In preferred embodiments, the topical skincare composition includes 1 to 3 wt. % of a reducing sugar (A) (e.g., dihydroxyacetone), 1 to 3 wt. % of an alkyl. sulfonate (B) (e.g., sodium 1-octanesulfonate, sodium 1-decanesulfonate, or both), 1 to 3 wt. % of a carrier benzyl alcohol), 2 to 8 wt. % of an organic solvent (D) (e.g., 1,3-propanediol, 1,2-propanediol, ethylene glycol, glycerin, 1,3-butanediol), 70 to 82 wt. % water (F), 0.001 to 1 wt. % of a preservative (H) (e.g., a mixture of methylparaben and ethylparaben), 0.001 to 0.2 wt. % of an acidulent (I) (e.g, citric acid), and 1 to 3 wt. % of a thickening agent (G) (e.g., SEPIPLUS™ S (hydroxyethyl acrylate/sodium acryloyldimethyl taurate copolymer, polyisobutene, PEG-7 trimethylolpropane coconut ether), each based on a total weight of the topical skincare composition.

In preferred embodiments, the topical skincare composition includes 1 to 3 wt. % of a reducing sugar (A) (e.g,, dihydroxyacetone), 1 to 3 wt. % of a mixture of an alkyl sulfonate (B) (e.g,, sodium 1-octanesulfonate, sodium 1-decanesulfonate, or both) and an aromatic sulfonate (E) (e.g,, sodium 2-naphthalenesulfonate, sodium p-toluenesulfonate, or both) in a weight ratio specified previously, 1 to 3 wt. % of a carrier (C) (e.g., benzyl alcohol), 2 to 8 wt ,% of an organic solvent (D) 1,3-propanediol, 1,2-propanediol, ethylene glycol, glycerin, 1,3-butanediol), 70 to 82 wt. % water (F), 0.001 to 1 wt. % of a preservative (H) (e.g., a mixture of methylparaben and ethylparaben), 0.001 to 0.2 wt. % of an acidulent (I) (e.g., citric acid), and 1 to 3 wt. % of a thickening agent (G) SEPIPLUS™ S (hydroxyethyl acrylate/sodium acryloyldimethyl taurate copolymer, polyisobutene, PEG-7 trimethylolpropane coconut ether), each based on a total weight of the topical skincare composition.

The topical skincare compositions herein can be prepared via any method known to those of ordinary skill in the art. By way of example, the topical skincare composition that contains water may be prepared by (i) mixing together all water soluble ingredients except for the tanning agent (e.g., the reducing sugar) in an appropriately sized vessel with water with optional heating (e.g., 40 to 90° C., preferably 50 to 85° C., more preferably 75 to 81° C.) and agitation until homogenous, (ii) in a separate vessel, mixing all oil phase ingredients, if any, with optional heating (e.g., 40 to 90° C., preferably 50 to 85° C., more preferably 75 to 81° C.) and stirring until homogeneous, (iii) mixing together the homogenous mixture from (i) with the homogenous mixture from (ii), if any, with optional heating (e.g., 40 to 90° C., preferably 50 to 85° C., more preferably 75 to 81° C.) and agitation until a homogenous aqueous composition or uniform oil in water emulsion is formed, (iv) cooling down the homogenous aqueous composition or uniform oil in water emulsion, and (v) once cooled, adding the tanning agent (e.g., the reducing sugar) to the homogenous aqueous composition or uniform oil in water emulsion, with optional agitation under condition similar to above, thereby forming the topical skincare composition. When present, the thickening agent may be added during step (i) or after step (iii) and before the addition of the tanning agent (i.e., before step (v)). Once cooled, the resulting topical skincare composition may then be filled into a desired packaging. The agitation may be provided by a propeller, such as a high shear mixer, and a high speed dissolver.

### Method for Self-Tanning

The present disclosure provides methods of adjusting a color of the skin by topically applying the topical skincare composition, in one or more embodiments, onto the skin of a subject.

In order to achieve an acceptable degree of coloration or tan a person who desires such coloration or tan can apply evenly an effective amount of the topical skincare composition over a desired body surface area for an effective application time. Thus, the topical skincare composition can be applied to provide subtle changes in skin color or more dramatic tanning effects.

The topical skincare composition can be topically applied to wet or dye skin. Preferably, the desired area of skin is cleaned, and/or exfoliated prior to application. During the application, the topical skincare composition may be directly spread on an outer skin using, e.g., the hands, an applicator such as a wipe, puff roller, or spray. The topical skincare compositions may be used as a single treatment to color the skin or applied in a progressive mariner so the skin tan becomes more intense on subsequent applications until a desired degree of coloration is reached.

The topical skincare composition may be applied to the desired area as needed, preferably 1 to 4 times daily, preferably 2 to 3 times daily. The application may be conducted for at least 1 day, preferably for at least 2 consecutive days, more preferably for at least 3 consecutive days, even more preferably for at least 4 consecutive days, and up to 14 consecutive days, preferably up to 7 consecutive days, more preferably up to 6 consecutive days, even more preferably up to 5 consecutive days. Alternatively, the application may be performed intermittently. Application times outside of these ranges may also be used to vary^{,} the degree of coloration, as desired.

The appearance of a color (e.g., the color of a skin) can be expressed in terms of its hue (color), lightness (brightness), and saturation (vividness). Hue is a major attribute of a color perception used for the denotation of “unique hues” (e.g., red, yellow, blue), which are considered completely different hues. Saturation (also called chroma or colorfulness) refers to is the “purity” of a specific hue. A highly saturated hue has a vivid, intense color, while a less saturated hue appears more muted and dull. With no saturation at all, the hue becomes a shade of gray. Lightness represents the brightness or darkness of a color perception. For example, an image with a higher lightness value reflects a greater quantity of light.

Quantifications of these color attributes (hue, saturation, lightness) can be performed using various color models including CIE (International Commission on Illumination) L*a*b* (CIELAB) color space model, CIE L*C*h° (CIEHCL) color space model, CIE XYZ model, RGB color model, and the like.

In some embodiments, the color of the skin herein is measured using the CIE L*a*b* model, where (i) L* denotes the lightness of the color which value runs from 0, representing black, to 100, representing white, (ii) a* denotes the reel/green value of the color, and (iii) b* denotes the yellow/blue value of the color. In some embodiments, the color of the skin herein is measured using CIE L*C*h° model, where (i) L* denotes the lightness of the color and is the same as L* of the CIE L*a*b* model, (ii) C* denotes saturation (chroma) with value starts from 0, representing no saturation, and reaches 60, representing full saturation, and (iii) h° denotes hue angle. As shown in the chromaticity diagram (FIG. 11), the hue angle of the color of the skin herein is expressed in degrees and starts at the +a* axis. Specifically, a hue angle of 0° would be red (+a*), a hue angle of 90° would be yellow (+b*). The color attributes of the skin herein can be measured by a spectrophotometer, such as CM-700d, CM-2500d, or CM-2600d spectrophotometer manufactured by Konica Minolta.

In some embodiments, the method disclosed herein produces a rich, deep, natural looking and long lasting tan on the skin by simultaneously preserving the original hue, as well as increasing saturation and darkness of the skin color.

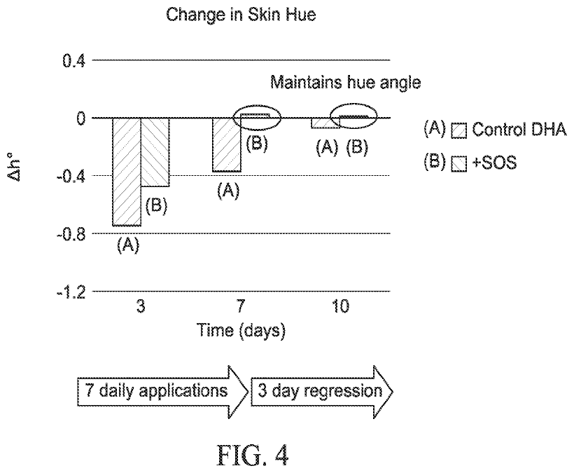

The method herein may preserve original hue of the skin after application of the topical skincare composition. In some embodiments, the method herein changes hue angle h° of the color of the skin by less than 2°, preferably less than about 1.5°, preferably less than about 1.2°, preferably less than about 1°, preferably less than about 0.8°, preferably less than about 0.6°, preferably less than about 0.4°, preferably less than about 0.3°, more preferably less than about 0.2°, even more preferably less than about 0.1°, yet even more preferably less than about 0.05°, compared to that prior to the topical application (see FIG. 4).

The method herein may darken the color of the skin after application of the topical skincare composition. In some embodiments, the method herein reduces lightness L* of the color of the skin by at least about 1%, preferably at least about 2%, preferably at least about 3%, preferably at least about 4%, more preferably at least about 5%, even more preferably at least about 6%, yet even more preferably at least about 7%, and up to about 20%, preferably up to about 15%, preferably up to about 12%, more preferably up to about 10%, even more preferably up to about 8%, compared to that prior to the topical application.

In preferred embodiments, the method herein simultaneously maintains the hue angle and reduces the lightness of the treated skin in accordance with the ranges specified above.

It is worth noting that the combination of a reducing sugar (A) (e.g., dihydroxyacetone, erythrulose), an alkyl sulfonate (B) (e.g., sodium 1-octanesulfonate, sodium 1-decanesulfonate) provides unexpected tanning results (darker, longer lasting (i.e., more fade-resistant), more natural looking coloration) compared to using only a reducing sugar.

For example, as shown in FIG. 4, using a reducing sugar alone significantly changes the hue angle of the treated skin, and in some cases actually increases the hue angle after prolonged applications (e.g., after 7 days application). This hue angle increase is typically the cause for the orange hue after self-tanning, which is undesirable to many consumers. However, combining the alkyl sulfonate (B) with the reducing sugar (A) effectively maintains the original hue angle, thus creating a natural looking tan to the treated skin.

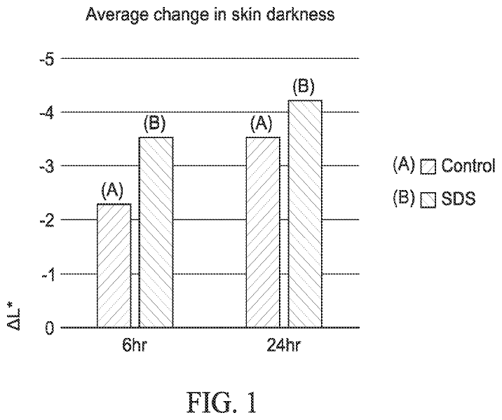

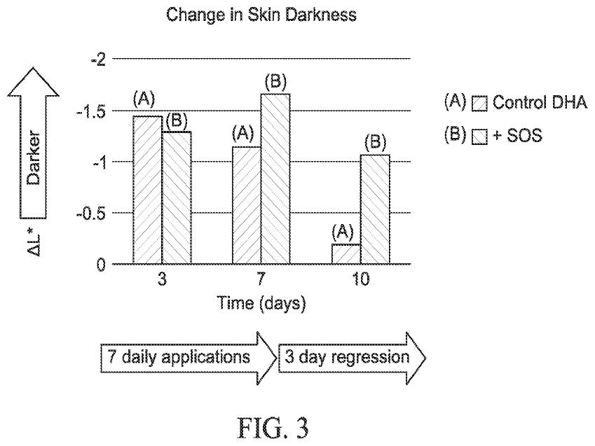

As shown in FIG. 1, within 24 hours after applications, using a reducing sugar alone leads to a reduction in skin lightness that is at least 15% less, preferably at least 20% less, more preferably at least 25% less, even more preferably 30% less, and up to 60% less, preferably up to 50% less, more preferably up to 40% less than using the combination of the reducing sugar (A) and the alkyl sulfonate (B). Further, as shown in FIG. 3, the artificial tan generated by this combination fades significantly slower than using a reducing sugar alone (e.g., less than about 40% color fade versus at least 80% color fade after 3 days regression).

The method herein may be utilized to adjust the color saturation of the skin. In some embodiments, the method herein increases saturation C* of the color of the treated skin by at least about 1%, preferably at least about 2%, preferably at least about 3%, preferably at least about 4%, more preferably at least about 5%, even more preferably at least about 6%, yet even more preferably at least about 7%, and up to about 20%, preferably up to about 15%, preferably up to about 12%, more preferably up to about 10%, even more preferably up to about 8%, compared to that prior to the topical application.

When an aromatic sultanate (E) is present in the topical skincare composition, the method herein may provide a synergistic tanning effect by shifting the original hue and further darkening the color of the skin. For example, as shown in FIGS. 6A-D, using a combination of the reducing sugar (A), the alkyl sulfonate (B), and the aromatic sulfonate (E) causes a darkening that is at least 25% more, preferably at least 28% more, more preferably at least 30% more, even more preferably at least 35% more, and up to 60% more, preferably up to 50% more, more preferably up to 40% more than using the reducing sugar (A) and the alkyl sulfonate (B) alone. In addition, using a combination of the reducing sugar (A), the alkyl sulfonate (B), and the aromatic sulfonate (E) causes a darkening that is at least 20% more, preferably at least 25% more, more preferably at least 28% more, even more preferably at least 30% more, and up to 50% more, preferably up to 40% more, more preferably up to 35% more than using the reducing sugar (A) and the aromatic sulfonate (E) alone. Further, the presence of the aromatic sulfonate (E) imparts an aesthetically pleasing red/bronze tone to the treated skin.

The examples below are intended to further illustrate the topical skincare compositions and are not intended to limit the scope of the claims.

### EXAMPLES

### Topical Skincare Compositions

Several example topical skincare compositions including comparative compositions used liar tanning performance evaluations are given in Examples 1-2 below. The amount of each component is expressed in terms of weight percentage relative to a total weight of 100%. DHA refers to dihydroxyacetone, available from EMD Millipore. SOS refers to sodium 1-octanesulfonate. SOS refers to sodium 1-decanesulfonate. SOBS refers to sodium dodecylbenzene sulfonate. SLSA refers to sodium lauryl sulfoacetate. NSA refers to sodium 2-naphthalenesulfonate, available from Sugai Chemical. BA refers to benzyl alcohol, available from Kalama, 1,3-butylene, methylparaben, ethylparaben, and SEPIPLUS™ S are each available from Oxea, Ueno, Sharon, and Seppic. * denotes the example is a comparative example in the tables below.

### Example 1

TABLE 1
Example topical skincare composition

| INCI Name | Amount (wt. %) |
|---|---|
| Water | 80.530 |
| 1,3-Butylene Glycol | 6.000 |
| Methylparaben | 0.300 |
| Ethylparaben | 0.150 |
| Benzyl Alcohol | 2.000 |
| Sodium 1-octanesulfonate | 2.000 |
| SEPIPLUS ™ S (hydroxyethyl acrylate/sodium | 2.000 |
| acryloyldimethyl taurate copolymer, polyisobutene, | |
| PEG-7 trimethylolpropane coconut ether) | |
| Water | 5.000 |
| Dihydroxyacetone | 2.000 |
| Citric Acid (50%) | 0.020 |

INCI = International Nomenclature of Cosmetic Ingredients

TABLE 2
Example topical skincare compositions for tanning evaluation

| | Exam- | Exam- | Exam- | Exam- | Exam- | Exam- | Exam- | Exam- | Exam- | Exam- | Exam- | Exam- | Exam- |
|---|---|---|---|---|---|---|---|---|---|---|---|---|---|
| | ple 1* | ple 2 | ple 3* | ple 4 | ple 5* | ple 6 | ple 7 | ple 8 | ple 9 | ple 10* | ple 11 | ple 12 | ple 13 |
| DHA | 5 | 5 | 2 | 2 | 3 | 3 | 3 | 3 | 3 | 5 | 5 | 5 | 5 |
| SDS | — | 2 | — | — | — | — | 2 | — | — | — | | — | — |
| SOS | — | — | — | 2 | — | 2 | — | — | — | — | 2 | — | 2 |
| SDBS | — | — | — | — | — | — | — | 2 | — | — | — | — | — |
| SLSA | — | — | — | — | — | — | — | — | 2 | — | — | — | — |
| NSA | — | — | — | — | — | — | — | — | — | — | — | 2 | 2 |
| BA | — | — | — | — | — | — | — | — | — | — | — | — | — |
| Glycerin | — | — | 7 | — | — | — | — | — | — | — | — | — | — |
| Butylene | 6 | 6 | — | — | — | — | — | — | — | — | — | — | — |
| Glycol | | | | | | | | | | | | | |
| Propanediol | — | — | — | 7 | — | — | — | — | — | — | — | — | — |
| Hydroxyethyl | 1 | 1 | 1.25 | 1.25 | 0.75 | 0.75 | 0.75 | 0.75 | 0.75 | 0.75 | 0.75 | 0.75 | 0.75 |
| cellulose | | | | | | | | | | | | | |
| Xanthan Gum | — | — | 0.1 | 0.1 | — | — | — | — | — | — | — | — | — |
| Methylparaben | 0.3 | 0.3 | 0.3 | 0.3 | 0.3 | 0.3 | 0.3 | 0.3 | 0.3 | 0.2 | 0.2 | 0.2 | 0.2 |
| Ethylparaben | 0.15 | 0.15 | 0.15 | 0.15 | 0.15 | 0.15 | 0.15 | 0.15 | 0.15 | 0.1 | 0.1 | 0.1 | 0.1 |
| Cetearyl | — | — | 3.5 | 3.5 | — | — | — | — | — | — | — | — | — |
| Alcohol | | | | | | | | | | | | | |
| Mineral Oil | — | — | 1.75 | 1.75 | — | — | — | — | — | — | — | — | — |
| Petrolatum | — | — | 1.75 | 1.75 | — | — | — | — | — | — | — | — | — |
| Dimethicone | — | — | 1.5 | 1.5 | — | — | — | — | — | — | — | — | — |
| Ceteareth-20 | — | — | 1.2 | 1.2 | — | — | — | — | — | — | — | — | — |
| Octyldodecyl | — | — | 1 | 1 | — | — | — | — | — | — | — | — | — |
| Myristate | | | | | | | | | | | | | |
| Ethylhexyl | — | — | 1 | 1 | — | — | — | — | — | — | — | — | — |
| Isononanoate | | | | | | | | | | | | | |
| Steareth-2 | — | — | 0.6 | 0.6 | — | — | — | — | — | — | — | — | — |
| BHT | — | — | 0.1 | 0.1 | — | — | — | — | — | — | — | — | — |
| Fragrance | — | — | 0.5 | 0.5 | — | — | — | — | — | — | — | — | — |
| Citric Acid | 0.005 | 0.005 | 0.01 | 0.01 | 0.005 | 0.005 | 0.005 | 0.017 | 0.037 | 0.006 | 0.002 | 0.008 | 0.002 |
| Caramel | — | — | 0.04 | 0.04 | — | — | — | — | — | — | — | — | — |
| Water | q.s. | q.s. | q.s. | q.s. | q.s. | q.s. | q.s. | q.s. | q.s. | q.s. | q.s. | q.s. | q.s. |
| Total | 100.00 | 100.00 | 100.00 | 100.00 | 100.00 | 100.00 | 100.00 | 100.00 | 100.00 | 100.00 | 100.00 | 100.00 | 100.00 |

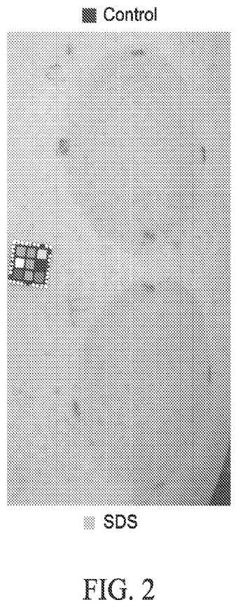

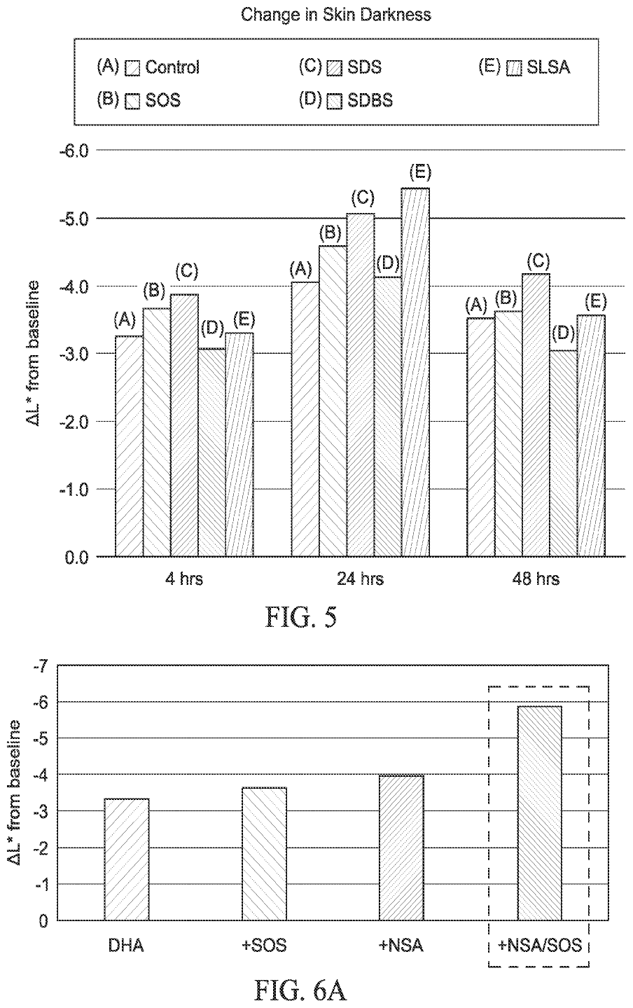

Examples 1* and 2 are compositions “control” and “SDS”, respectively tested in FIGS. 1 and 2; Examples 3* and 4 are compositions “control DHA” and “SOS”, respectively tested in FIGS. 3-4; Examples 5*, 6, 7, 8, and 9 are compositions “Control”, “SOS”, “SDS”, “SDBS”, and “SLSA”, respectively tested in FIG. 5; and Examples 10*, 11, 12, and 13 are compositions “DHA”, “+SOS”. “+NSA”, and “+NSA/SOS”, respectively tested in FIGS. 6A-D.

### Preparation Methods

An exemplary process for preparing the example topical skincare composition is as follows:

Part A: Deionized water and 1,3-butylene glycol were first added to a main vessel and mixed thoroughly. The main vessel was heated to a temperature of 75-81° C. When the temperature of the vessel reached 75° C., parabens, BA, and SOS were added to the main vessel and mixed until clear. The heat was then turned off SEPIPLUS™ S was added to the main vessel and mixed for about 20 minutes. Increasing agitation could be optionally applied as the mixture thickened after the addition of SEPIPLUS™ S. The main vessel was cooled to a temperature of below 40° C., thereby forming the Part A.

Part B: Deionized water and DHA were added to a separate vessel and mixed until clear to form the Part B.

After Part A was cooled to a temperature below 40° C., part B was added to Part A to form part AB, which was mixed for about 5 minutes.

Finally, citric acid was added to part AB to adjust the pH to 3.5-4.0, thereby forming the topical skincare compositions.

Procedure for Collecting Stratum Corneum from Volunteers' Heels

(i) cleaned up heels with cotton swabs soaked in alcohol in order to remove residues on the skin surface;

(ii) waited for 5-10 min to make sure the skin is dry;

(iii) harvested the stratum corneum with a heel buffer* onto a piece of aluminum foil (*preferably using the following heel buffer by Panasonic:
https://www.amazon.com/Panasonic-ES2502P-Electric-Heel-Buffer/dp/B001CS69NQ);

(iv) mixed up the collected flakes, especially if the flakes were collected from more than one volunteer; and

(v) dried the flakes under room temperature and humidity conditions for more than 3 hours.

### Determination of Relative Keratin Swellability

(i) added 20 mg of dried, stratum corneum powder to an appropriate number of NMR tubes (Wilmad-LabGlass WG-1000-7, inner diameter 4,1 mm) to prepare the test sample(s) and a control sample;

(ii) set the NMR tubes in a tube rack, labeled each tube or location in the rack, placed the rack in a light box, and captured a photograph with a digital camera;

(iii) prepared the test sample(s) by making an aqueous solution(s) containing deionized water and 2% (w/w %) of a test compound, and adding 1.5 mL of the solution to the appropriate labeled NMR tube;

(iv) prepared the control sample by adding 1.5 mL of deionized water to the appropriate labeled NMR tube;

(v) gently shook each NMR tube sample manually, but without inverting, for about 1 minute to disperse the stratum corneum powder in the solution uniformly;

(vi) sealed the NMR tubes, set in a tube rack, and placed the rack in a 40° C. controlled temperature room for 1 hour;

(vii) thereafter, placed the tube rack with all samples in a light box and captured a photograph with a digital camera;

(viii) measured the height of the stratum corneum powder of each sample using image analysis capabilities in ImageJ (NIH) software; and

(ix) calculated the keratin swellablity of the est compound(s) according to the following formula (A):

$$Relative Keratin Swellability , {S}_{relative} = \frac{Hsolution - Hdry}{Hwater - Hdry} \qquad ( A )$$

where Hₛₒₗᵤₜᵢₒₙ is the height of the stratum corneum when exposed to a test solution containing 2% (w/w %) of a test compound in deionized water, H_{dry} is the height of the stratum corneum before adding a test solution or deionized water, and H_{water} is the height of the stratum corneum in deionized water.
Determination of Relative α-helix/β-Sheet Index Ratio

(i) 20 mg dried stratum corneum powder was added to an appropriate number of NMR tubes (Wilmad-LabGlass WG-1000-7, inner diameter 4.1 mm) to prepare the test sample(s) and a control sample;

(ii) set the NMR tubes in a tube rack and labeled each tube or location in the rack;

(iii) prepared the test sample(s) by making an aqueous solution(s) containing deionized water and 2% (w/w %) of a test compound, and adding 1.5 mL of the solution to the appropriate labeled NMR tube;

(iv) prepared the control sample by adding 1.5 mL of deionized water to the appropriate labeled NMR tube;

(v) gently shook each NMR tube sample manually, but without inverting, for about 1 minute to disperse the stratum corneum powder in the solution uniformly;

(vi) sealed the NMR tubes, set in a tube rack, and placed the rack in a 40° C. controlled temperature room for 96 hours;

(vii) thereafter, gently shook each NMR tube sample manually, but without inverting, for about 1 minute;

(viii) removed the supernatant, added 1.5 mL of deionized water to the tube, and leave it at room temperature for more than 15 min;

(ix) repeated step (viii) two additional times;

(x) the stratum corneum was raked out with a spatula, and transferred into a clean, 8-dram vial;

(xi) dried the stratum corneum under room temperature and humidity conditions for more than 12 hours;

(xii) thereafter, analyzed each dried sample by FT-IR (Nicolet iS50, Thermo Fisher Scientific Inc., MA, USA) equipped with a ZeSe ATR accessory and a DIGS detector. Scan 32 times in the wavenumber range 4000 to 650 cm⁻¹ at a resolution of 4 cm⁻¹; and

(xiii) analyzed the spectra with a house-made software (S. Takada, S. Naito, J. Sonoda, Y. Miyauchi. Non-invasive in vitro measurement of natural moisturizing factor content in stratum corneum of human skin by attenuated total reflection infrared spectroscopy. Applied Spectroscopy 2012; 66: 26-32, incorporated herein by reference in its entirety) that provides the α-helix/β-sheet index corresponding to the protein secondary structure change (H, Takahashi H. Tsuji M. Minami-Hori Y. Miyauchi H. Iizuka, Defective barrier function accompanied by structural changes of psoriatic stratum corneum, The Journal of Dermatology 2014; 41: 144-148, incorporated herein by reference in its entirety). The relative α-helix/β-sheet index ratio of the test sample was calculated via dividing the α-helix/β-sheet index of the test sample by that of the control sample.

### Topical Skincare Composition Evaluation Methods

### Skin Test

The example composition was applied to the skin in an amount of 2 mg/cm². The color parameters of the skin (e.g., lightness L*, hue angle h°)were measured before the application, and at various time points after the test product application specified in each test, followed by calculating the changes of each parameter (ΔL*, (ΔC*, Δh°) compared to that before the application. Specifically, CM-2600d manufactured by Konica Minolta, Inc. was used as a colorimeter.

The skin tests could be performed as a consumer home-use study. For example, the data of FIGS. 3 and 4 were collected by the following method. Consumers applied one lotion product containing DHA alone (“Control DHA”), or DHA and SOS (“+SOS”), to their arras and legs once daily for 7 days. Color measurements were taken on the arms and legs at baseline (Day 0), Day 3, and Day 7. Starting on Day 7, the panelists stopped using the test product. Additional color measurements on the arms and legs were performed on Day 10.

The present disclosure also contemplates other embodiments “comprising”, “consisting of” and “consisting essentially of”, the embodiments or elements presented herein, whether explicitly set forth or not.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A topical skincare composition, comprising:
(A) about 0.1 to 30 wt. % of a reducing sugar relative to a total weight of the topical skincare composition;
(B) an optionally substituted C₈₋₁₂ alkyl sulfonate; and
(C) a carrier.

2. The topical skincare composition of claim 1, wherein the optionally substituted C₈₋₁₂ alkyl sulfonate (B) is an unsubstituted alkyl sulfonate.

3. The topical skincare composition of claim 1, wherein the optionally substituted C₈₋₁₂ alkyl sulfonate (B) is an alkali metal alkyl sulfonate salt.

4. The topical skincare composition of claim 1, wherein a weight ratio of the optionally substituted C₈₋₁₂ alkyl sulfonate (B) to the reducing sugar (A) ((B):(A)) is 1:20 to 20:1.

5. The topical skincare composition of claim 1, wherein the reducing sugar (A) is dihydroxyacetone, erythrulose, or both.

6. The topical skincare composition of claim 1, wherein the carrier (C) comprises an aromatic alcohol.

7. The topical skincare composition of claim 1, wherein the carrier (C) comprises benzyl alcohol.

8. The topical skincare composition of claim 1, further comprising (D) an organic solvent.

9. The topical skincare composition of claim 8, wherein the organic solvent (D) is at least one selected from the group consisting of 1,3-propanediol, 1,2-propanediol, and 1,3-butanediol.

10. The topical skincare composition of claim 1, further comprising (E) an aromatic sulfonate, wherein the aromatic sulfonate (E) has a topological polar surface area (tPSA) of less than 100 Å².

11. The topical skincare composition of claim 10, wherein the aromatic sulfonate (E) is an optionally substituted phenyl sulfonate, an optionally substituted naphthyl sulfonate, or both.

12. The topical skincare composition of claim 10, wherein a weight ratio of the alkyl sulfonate (B) to the aromatic sulfonate (E) ((B):(E)) is 1:100 to 100:1.

13. A method of darkening color and maintaining hue of the skin of a subject, the method comprising:
topically applying the topical skincare composition of claim 1 onto the skin of the subject.
wherein the topical application reduces a lightness L* of the color by at least 10%, and changes a hue angle h° of the color by less than 2°, each compared to those prior to the topical application.

14. A method of darkening color of the skin of a subject, the method comprising:
topically applying the topical skincare composition of claim 1 onto the skin of the subject,
wherein the topical application reduces a lightness L* of the color by at least 10% compared to that prior to the topical application.

15. A method of adjusting color saturation of the skin of a subject, the method comprising:
topically applying the topical skincare composition of claim 1 onto the skin of the subject,
wherein the topical application increases a saturation C* of the color by at least 10% compared to that prior to the topical application.

16. A collection of topical skincare products for retail sale, the collection comprising:
(a) a first topical skincare composition that comprises:
(A) about 0.1 to 30 wt. % of a reducing sugar relative to a total weight of the topical skincare composition;
(B) an optionally substituted C₈₋₁₂ alkyl sulfonate; and
(C) a carrier,
wherein a weight ratio of the optionally substituted C₈₋₁₂ alkyl sulfonate (B) to the reducing sugar (A) ((B):(A)) is 1:20 to 20:1, and
(b) a second topical skincare composition that comprises:
(A) about 0.1 to 30 wt. % of a reducing sugar relative to a total weight of the topical skincare composition;
(B) an optionally substituted C₈₋₁₂ alkyl sulfonate; and
(C) a carrier,
wherein a weight ratio of the optionally substituted C₈₋₁₂ alkyl sulfonate (B) to the reducing sugar (A) ((B):(A)) is 1:20 to 20:1,
wherein a content of the reducing sugar (A) present in the first topical skincare composition (a) is less than that of the reducing sugar (A) present in the second skincare composition (b), and wherein the contents are each relative to total weights of the first and second topical skincare compositions.

17. The collection of claim 16, wherein the first topical skincare composition (a) and the second topical skincare composition (b) are separately packaged.
